(19) United States
(12) Patent Application Publication
(10) Pub. No.: US 2003/0082148 A1
(43) Pub. Date: May 1, 2003

(54) Methods and device compositions for the recruitment of cells to blood contacting surfaces in vivo

(76) Inventors: Florian Ludwig, Atherton, CA (US); A. Adam Sharkawy, Union City, CA (US)

Correspondence Address: BOZICEVIC, FIELD & FRANCIS LLP, 200 MIDDLEFIELD RD, SUITE 200, MENLO PARK, CA, 94025, US

(21) Appl. No.: 10/286,385

(22) Filed: Oct. 31, 2002

Related U.S. Application Data
(60) Provisional application No. 60/334,621, filed on Oct. 31, 2001.

(51) Int. Cl. C12N 5/08; A61F 2/04; A61K 45/00

(52) U.S. Cl. 424/93.7; 435/372; 623/23.64

(57) ABSTRACT

Methods and compositions for recruiting cells circulating in the blood stream of a subject to a blood contacting surface, and in particular, devices and methods for recruiting endothelial cells to a blood contacting surface of a prosthesis as well as engineering a self-endothelializing graft in vivo by recruitment of circulating endothelial progenitor cells (EPCs) to form a neo-endothelium on a prosthetic structure.

This application claims benefit under 35 U.S.C. §119(e) of U.S. Provisional Application No. 60/334,621, filed Oct. 31, 2001, the entire disclosure of which is herein incorporated by reference.

### FIELD OF THE INVENTION

This invention pertains to the in vivo recruitment of cells to a blood contacting surface, including inventions to devices and methods for the recruitment of endothelial progenitor cells to a blood contacting surface of a prosthesis and developing vascular structures via in vivo endothelialization.

### LITERATURE

Barrera, D., E. Zylstra, et al. (1993). J Am Chem Soc 115: 11010-11; Belden, T. and S. Schmidt (1982) Trans Am Soc Artif Intern Organs. 28: 173-7; Bhattacharya, V., P. A. McSweeney, et al. (2000) Blood 95(2): 581-5; Budd, J. S., J. Hartley, et al. (1992) British Journal of Surgery 79: 1151-1153; Cook, A. D., J. S. Hrkach, et al. (1997) J Biomed Mater Res 35(4): 513-23; Cook, A. D., U. B. Pajvani, et al. (1997) Biomaterials 18(21): 1417-24; Greisler, H. P., D. Petsikas, et al. (1993) J Biomed Mater Res 27(7): 955-61; Hernandez, D. A., L. E. Townsend, et al. (2000) Am Surg 66(4): 355-8; discussion 359; Herring, M., S. Baughman, et al. (1984) Surgery 96(4): 745-55; Hrkach, J., N. Lotan, et al. (1995) Macromolecules 28: 4736-39; Hubbell, J. A., S. P. Massia, et al. (1991) Biotechnology (N Y) 9(6): 568-72; Hussain, S. and G. Long (1991) Journal of Surgical Research 51(1): 33-39; Kadletz, M., H. Magometschnigg, et al. (1992) J Thorac Cardiovasc Surg 104(3): 736-42 Kang, I. K., O. H. Kwon, et al. (1996) Biomaterials 17(8): 841-7; Li, J. M., M. J. Menconi, et al. (1993) Cardiovasc Surg 1(4): 362-8; Massia, S. P. and J. A. Hubbell (1990) Anal Biochem 187(2): 292-301; Massia, S. P. and J. A. Hubbell (1990) Ann NY Acad Sci 589: 261-70; Massia, S. P. and J. A. Hubbell (1992) J Biol Chem 267(20): 14019-26; Meinhart, J., M. Deutsch, et al. (1997) Asaio J 43(5): M515-21; Poole-Warren, L. A., K. Schindhelm, et al. (1996) J Biomed Mater Res 30(2): 221-29; Poot, A. and A. Beugeling (1992) Biomaterial-Tissue Interfaces. P. J. D. e. al, Elsevier Science Publishers. 10: 253-263; Schmidt, S. P., T. J. Hunter, et al. (1985) J Vasc Surg 2(2): 292-7; Shindo, S., A. Takagi, et al. (1987) J Vasc Surg 6(4): 325-32; Takahashi, T., C. Kalka, et al. (1999) Nat Med 5(4): 434-8; Thomson, G. J. L. and R. K. Vohra (1991) Surgery 109(1): 20-27; Ushida, T. (1992) Biomaterial-Tissue Interfaces, Elsevier Science Publishers B.V. 10: 99-103; Wang, Z., W. Du, et al. (1990) Journal of Vascular Surgery 12(2): 168-79; Zilla, P. and M. Deutsch (1994) Journal of vascular Surgery 19(3): 540-8; Zilla, P. and R. Fasol (1989) Surgery 105: 512-22. U.S. Pat. Nos. 6,159,531; 5,599,703; 5,880,090; 5,858,782; 6,049,026; 6,096,525; 5,977,252; 5,912,177; 5,628,781.

### BACKGROUND OF THE INVENTION

One of the major challenges in the development of blood contacting implant surfaces is to overcome the risk of acute thrombosis and chronic instability—such as calcification—of the implant surface. Surfaces of prostheses which are implanted as part of the circulatory system, such as heart valves and synthetic grafts, and in particular small diameter conduits used as vessel bypass grafts (such as for bypassing a blocked coronary artery), are the crucial factor governing the functionality and patency rates of these synthetic prosthesis. Poor blood compatibility of these surfaces is almost always the predominant reason for the limitations of these implants, such as the loss of heart valve functionality over time or poor patency rates in small diameter conduits due to acute thrombosis or intimal hyperplasia. Attempts to modify the surfaces of synthetic grafts to overcome the patency problems associated with thrombosis or intimal hyperplasia have generally shown poor long-term outcomes, as these surfaces are unable to maintain a sustained anti-thrombogenic bioactivity (Hayward, Johnston et el, 1985; Hayward, Durrani et al. 1986; Hall, Bird et al, 1989; Segesser, Olah et al. 1993; Walpoth, Rogulenko et al. 1998; Wagner, Deibl et al. 1999).

One surface modification approach which has been utilized for blood contacting implants such as synthetic grafts is “endothelial seeding”. In vitro endothelial seeding utilizes viable endothelial cells which are seeded onto the blood contacting surface of a prosthesis such as the lumen surface of a vascular graft to mimic the surface of natural blood vessels. This surface modification technique aims to produce a confluent, biologically active surface of viable endothelial cells which by definition, is anti-thrombogenic (Graham, Burkel et al. 1980; Graham, Vinter et al. 1980; Pasic and Mulle-Cilause 1996; Williams and Jarrdl 1997; Bowlin and Rittgers 1997; Bos, Scharenborg et al. 1998; Bos, Scharenborg et al. 1999). For endothelial seeding, autologous endothelial cells are harvested from the graft recipient to prevent immunogenic reaction. The endothelial cells can be seeded directly onto the lumen surface of the graft or after expansion in a cell culture. The synthetic grafts which are seeded by in vitro attachment of endothelial cells can be made of inert substances and/or biodegradable/resorbable materials which, after endothelial seeding, can be implanted in the graft recipient (Greisler, Joyce et al. 1992; Petsikas et al 1993; Shum-Tim, Stock et al. 1999; Greenwald and Berry 2000; U.S. Pat No. 5,916,585, Cook; U.S. Pat No. 6,238,687, Mao; U.S. Pat. No. 5,968,092, Buscemi; Huynh et al. Nature Biotech. 17(11): 1083-1086, 1999). Although “endothelial seeding” is an improvement, the need to harvest, expand, and seed endothelial cells brings with it additional complications. To obtain a sufficient amount of cells to seed a synthetic graft, endothelial cells must be isolated from the graft recipient, purified from a mixture of different cells and then expanded in vitro to produce enough endothelial cells for seeding the graft. Furthermore, the retention of endothelial cells on the surface of the graft is often insufficient, resulting in poor patency rates.

Techniques aimed to improve the retention of endothelial cells on vascular grafts have been reported. For example, the use of physical force to apply endothelial cells to graft surfaces is described in U.S. Pat. No. 5,037,378 to Muller et al. In another approach, U.S. Pat. No. 4,804,382 to Turina and Bittman describes the application of endothelial cells to a semi-permeable membrane in which the pores are filled with aqueous gels to allow endothelial cell coverage. Another method to prevent endothelial loss after seeding is to modify the graft lumen surface to make it sufficiently adhesive for endothelial cells. Surface modification methods include the interstitial deposition of protein glues or matrices, the adsorption of proteins to the graft surface, and the covalent immobilization of adhesion-promoting ligands, peptides, or proteins onto functional groups introduced by chemical modification or gas plasma treatment (Ramsey, Hertl et al. 1984; Radomski, Jairell et al, 1987; Seeger and Klingmann 1988; Conforti, Zanetti et al. 1989; Kaehler, Zilla et al. 1989; Matsuda, Kondo et al. 1989; Muller-Gluser and Zilla 1993; Terlingen, Brenneisen et al. 1993; Dang and Chiu 2000; Nishibe, Oduda et al. 2000; Patnaik 2000; Lavik, Hrkach et al. 2001). Another method for preventing the loss of endothelial cells and for improving patency rates of synthetic grafts involves using shear stress to pre-condition the endothelial layer of a synthetic graft. Unfortunately, so far none of these approaches have proven entirely successful. Furthermore, this preconditioning process is very lengthy, substantially increasing the necessary preparation time of the synthetic graft (Ballermann and Ott 1999; Ott and Ballermann 1995; Dardik and Liu 1999).

While substantial progress has been made on the road to a stably endothelialized implant surface over the last decade, all attempts at the in vitro engineering of endothelialized implants share a significant shortcoming common to all in vitro tissue engineering: the need to harvest, expand and culture the patient's autologous cells requires lengthy preparation time, a two step intervention—for cell harvest and subsequently for prosthesis implantation—and last not least bares the risk of failure through cell culture contamination. The disadvantages of the aforementioned synthetic implant techniques demonstrate the need for a method of making synthetic implant surfaces which offer long term viability and reliability, and which has the advantages of surface modification by endothelial cell adhesion without the cumbersome and time consuming in vitro processes associated with such surface modifications.

Recently, agents capable of increasing the levels of circulating endothelial cell precursors have been utilized in methods for enhancing the endothelialization of synthetic vascular grafts. Administering cytokines such as G-CSF and GM-CSF have been useful in enhancing the endothelialization of synthetic vascular grafts (U.S. Pat. No. 5,880,090). However, these grafts lose resilience and become stiff after implantation for four weeks or longer. In grafts implanted longer than four weeks, osteoblasts, osteocytes, and microcalcifications were found which affected the long-term utility of such grafts.

This invention discloses methods for overcoming these limitations by facilitating in vivo tissue engineering through the recruitment of circulating cells to graft and/or prosthesis surfaces. In addition, the invention discloses methods which ensure the permanent population and modification of implant surfaces by said cells. In one specific embodiment, the recruitment of endothelial progenitor cells to implant surfaces facilitates the endothelialization and hence blood compatibilization of these surfaces.

Furthermore, this invention discloses methods which utilize the targeting of genetically modified cells to specific surfaces as a way of achieving a localized, sustained substance production and release local to said surface.

### SUMMARY OF THE INVENTION

The present invention provides methods and devices which allow for the targeting of specific cells circulating in the blood stream of a subject to a blood contacting surface, in vivo. The recruitment of target cells to the blood contacting surface, comprises providing a blood contacting surface positioned in the blood stream of a subject, the blood contacting surface configured to actively recruit target cells circulating in the blood stream of the subject to the blood contacting surface.

The blood contacting surface, in certain embodiments may be a surface of a prosthesis implanted into the subject, a surface of a pre-existing medical device or a surface of a blood vessel of the subjects. Recruiting the target cells to the blood contacting surface may comprise magnetically attracting the target cells to the blood contacting surface, and in other embodiments, recruiting the target cells comprises introducing ligands onto the blood contacting surface, the ligands having an affinity for the target cells. Introducing the ligand to the blood contacting surface may comprise coating the ligand onto the blood contacting surface. The coating of the blood contacting surface may be completed either in vitro or in vivo. Recruiting the target cells to the blood contacting surface may also comprise modifying the target cell to enhance the affinity of interaction with the blood contacting surface.

The blood contacting surface in certain embodiments, is modified by the adherence, spreading, and/or differentiation of the target cells over the blood contacting surface. Examples of prosthetic devices comprising a blood contacting surface when positioned in the circulatory system of a subject include but are not limited to stents, heart valves, artificial hearts, arterial and venous blood vessel prostheses, anastomotic devices and vascular and capillary structures of organ prostheses. The prosthetic devices include biocompatible non-degradable implants as well as implants which are made from biodegradable materials. Targeted cells include cells naturally found within the blood stream as well as cells introduced into the blood stream of a subject. Target cells may be modified to adhere or be attracted to the blood contacting surface. Target cells may also have been genetically modified to express a specific substance once adhered to the blood contacting surface.

Target cells of the invention include but are not limited to progenitor cells, red blood cells, mononuclear cells, macrophages, cells of the immune system such as T helper cells, platelets and progenitor cells, wherein the progenitor cells comprise endothelial progenitor cells. In certain embodiments, methods of the invention comprise introducing the target cells into the bloodstream of the subject. The target cells introduced into the blood stream comprise autologous cells and donor cells. The target cells may be harvested from bone marrow or fat tissue and/or cultured in vitro. Introducing the target cells into the blood stream may further comprise injecting the target cells into the bloodstream of the subject.

The present invention provides prostheses comprising a support member having a blood contacting surface capable of forming a magnetic interaction with a target cell circulating in the blood stream of a subject. In one embodiment, the blood contacting surface is configured to magnetically attract a magnetically modified target cells in vivo. In other embodiments, the blood contacting surface is configured to present a ligand which binds to a cell's surface molecule of the target cell. In certain embodiments, the prosthesis comprises, a first layer of a cross-linked polymeric compound coated onto the blood contacting surface of the support member and a second layer coated on the first layer, the second layer comprising at least one ligand having an affinity for a targeted cell in vivo.

One embodiment of the present invention provides methods and devices which allow for the establishment of a bioactive, anti-thrombogenic prosthesis by the in vivo recruitment of endothelial progenitor cells (EPC) circulating in the blood of a graft recipient to the blood contacting surface of the graft prosthesis or medical implant. Subsequently, the differentiation of the adhered progenitor cells allows the formation of a functioning endothelium. The present invention is useful in endothelializing the surface of blood contacting prosthetic devices in vivo.

One of the challenges in the use of tissue engineered organs is the establishment of adequate blood supply to the tissue of the organ. Similar to natural tissue, this requires the existence of a vascular network within the structure of the engineered tissue. These networks require an endothelial lining to stabilize them against thrombus formation. In order to endothelialize these networks, endothelial cells or their progenitor cells have to be recruited to the lumenal (blood contacting) surfaces of the vascular network. The present invention provides methods for the recruitment of cells to a blood contacting surface of the prosthesis, post-implantation, by designing the blood contacting surface of the implant to retain endothelial progenitor cells from the blood flow through ligand-receptor interaction, magnetic interaction and other physical and chemical interactions disclosed below.

The present invention is also useful for forming vessel structures used to bypass at least a portion of a native vessel. The present invention is suitable for the formation of blood vessels for bypassing blockages or occlusions within native coronary arteries, such as those suffering from atherosclerosis. As pointed out above, bypassing of blockages or occlusions may also be achieved by the connection of two distinct native vessels.

The subject methods comprise providing a scaffolding (prosthesis) having a surface exposed to the recipient's circulating blood, implanting the prosthesis at the desired location within the recipient's body, recruiting circulating target cells such as endothelial progenitor cells (EPCs) from the implant recipient's blood to a blood contacting surface of the scaffolding to form a neo-endothelium. In certain embodiments of the invention the subject methods may further comprise encapsulating an exterior surface of the scaffolding with vascular tissue to form a hemostatic adventitial structure. In certain embodiments the scaffolding may be biodegradable and the subject methods may also comprise degrading the biodegradable scaffolding under in vivo conditions within a time-frame that allows the neo-endothelium and the adventitial structure to form a functional neo-vessel. The method may also comprise retaining and spreading the recruited progenitor cells to facilitate differentiation of the progenitor cells into functioning endothelial cells. The invention may further comprise increasing the number of circulating progenitor cells in an implant subject by mobilizing the target cells from tissue into the blood circulation.

The endothelializable prosthesis (scaffolding) may comprise biodegradable and/or non-biodegradable materials. The prosthesis is implanted in the recipient to allow at least one surface of the prosthesis to be exposed to blood flow from the recipient's circulatory system. The prosthesis may be configured to operate as a temporary or permanent structure.

Pluripotent stem or progenitor cells found in tissues such as bone marrow, umbilical cord, and peripheral blood are potential sources of precursor cells for a variety of cell types, including endothelial cells (Asahara, Murohara et al. 1997; Long and Pipia 1999; Murohara, Ideda et al. 2000; Boyer, Townsend et al. 2000). These endothelial progenitor cells are circulating in peripheral blood and have been shown to play a role in wound repair as well as angiogenesis (Shi, Wu et al 1994; Asahara, Murohara et al. 1997; Shi, Rafii et al. 1998; Asahara, Takahashi et al. 1999; Boyer, Townsend et al. 2000). Progenitor cells have also been shown to differentiate into fully functional endothelial cells in vivo and in vitro and can be mobilized from the bone marrow by treatment with VEGF, G-CSF, GM-CSF or by ischemia (Segal and Bagby 1988; Gehling, Ergun et al. 2000; Hammond, Shi et al. 1999; Kalka, Masuda et al. 2000; Kalka, Tehrani et al. 2000).

The recruitment of target cells to the blood contacting surface and the spreading thereof, may be accomplished by functionalizing the surface and/or coating (adsorbing) the surface to be functionalized with compounds or groups that specifically interact with target cell surface molecules. Compounds which aid in the differentiation of target cells, such as EPCs to functional endothelial cells may also be adsorbed or functionally attached to the surface of the blood contacting surface. The endothelializable surface of the scaffolding may be modified to promote progenitor cell adhesion thereto. Such surface modification may be accomplished with ligands recognizing endothelial progenitor cells. The ligands may bind, for example, to a protein or molecule present on the surface of the endothelial progenitor cell, such as CD34, CD133, KDR (VEGFR-2), VE-Cadherin, E-selectin, αᵥβ₃, lectins, or other cell surface molecules. Alternatively, the recruitment of endothelial progenitor cells to the prosthesis surface may be achieved by the introduction of specific, bifunctional molecules onto the EPC surface by systemic medication, where one functionality of the bifunctional molecules binds to EPC surface molecules such as mentioned above and the other functionality adheres to the prosthesis surface, either through ligands immobilized at this surface which bind to this functionality or through hydrophobic, electrostatic or magnetic attraction of the functionality to the surface. The endothelializable surface may further comprise a compound or compounds which promote differentiation of the progenitor cells into endothelial cells. Such compounds may comprise, for example, VEGF, FGF or other growth factors or a combination thereof. The endothelializable surface may further comprise a compound or compounds which promote the stable adhesion and spreading of EPCs or the endothelial cells which differentiate from them. Such compounds may comprise peptides, proteins or amino acid sequences, in particular amino acid sequences found in basement membrane proteins and the native extracellular matrix of endothelial cells. The endothelializable surface may further comprise a compound or compounds which are released over a period of time which target specific blood circulating molecules, including cells, which promote the formation of a functional endothelium.

Encapsulation of the scaffold (medical implant) may be carried out as described in U.S. patent application Ser. No. 09/863,198 and entitled “Methods and Devices for in situ Formation of Vascular Structures Suitable for use as Blood Vessels”, filed May 22, 2001, herein incorporated by reference. In the aforementioned patent application, the inventors demonstrated that a vascular structure is formed by encapsulating an implanted synthetic silicone mandrel. The vascular tissue conduit formed around the mandrel is capable of being completely hemostatic along its length and of supporting physiological pressures after removal of the mandrel. The prosthesis (graft scaffolding) in one embodiment of the present invention allows for the in vivo generation of a vascular tissue around the prosthetic structure. When the material of the prosthesis is biodegradable, the vascular tissue will ultimately replace the biodegradable prosthesis.

These and other objects, advantages, and features-of the invention will become apparent to those persons skilled in the art upon reading the details of the methods and devices of the present invention which are more fully described below.

### BRIEF DESCRIPTIONS OF THE DRAWINGS

FIGS. 1A-C schematically illustrates a procedure for the functionalization of a blood contacting surface.

FIGS. 2A-B schematically illustrates an alternative procedure for functionalizing a blood contacting surface for binding targeted cells.

FIGS. 3A-B are schematic illustrations of a multi-functional surface configuration for a blood contacting surface.

FIG. 4 is a plan view of one embodiment of a prosthesis of the present invention.

FIG. 5 is a cross-sectional view of the prosthesis shown in FIG. 4.

FIGS. 6A-D schematically illustrates the stages of forming a neo-vessel using the biodegradable scaffolding of the present invention.

### DETAILED DESCRIPTION OF THE INVENTION

The present invention provides devices and methods for recruiting circulating cells to a blood contacting surface, in particular, devices and methods for actively recruiting progenitor cells (e.g. endothelial progenitor cells) circulating in the bloodstream of a subject to the blood contacting surface.

The present invention provides devices and methods for recruiting circulating cells to a blood contacting surface of a prosthesis, as well as devices and methods for engineering an in vivo self-endothelializing prosthesis by targeting and recruitment of circulating endothelial progenitor cells (EPCs) from the blood stream to an endothelializable surface (blood contacting surface) of a prosthetic device or scaffolding.

Before the devices and methods of the present invention are described, it is to be understood that this invention is not limited to any particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It should be noted that as used herein and in the appended claims, the singular forms “a”, “and”, and “the” include plural referents unless the context clearly dictates otherwise. Thus, for example, reference to “an endothelial cell” includes a plurality of such cells and reference to “the functional group” includes reference to one or more functional groups and equivalents thereof known to those skilled in the art, and so forth.

The publications discussed herein are provided solely for their disclosure prior to the filing date of the present application. All publications mentioned herein are incorporated by reference to disclose and describe the methods and/or materials in connection with which the publications are cited. Nothing herein is to be construed as an admission that the present invention is not entitled to antedate such publication by virtue of prior invention. Further, the dates of publication provided may be different from the actual publication dates which may need to be independently confirmed.

### DEFINITIONS

The term “subject”, as used herein defines any mammalian subject for whom diagnosis or therapy is desired, particularly humans. Other subjects may include cattle, dogs, cats, guinea pigs, rabbits, rats, mice, horses, and so on.

The term “recruiting”, “recruitment” and their derivatives are used herein to refer to specifically selecting a blood circulating target cell or modified target cell, and providing a blood contacting surface which is configured to adhere the blood circulating target cells to the blood contacting surface. Recruiting (attracting and binding) the target cells to the blood contacting surface may include magnetic attraction, electrostatic attraction, inter-molecular binding, ligand binding or a combination thereof, between the target cell and the blood contacting surface.

The term “targeting”, “target cell” and their derivatives are used herein to refer to a cell circulating in a subject's blood stream which has been preferentially selected to interact with the blood contacting surface, and in particular a blood contacting surface of the implanted prosthesis. Exemplary target cells include but are not limited to progenitor cells, endothelial cells, endothelial progenitor cells, platelets, cells of the immune system, such as T-helper cells, macrogphages, bacterium and red blood cells. Target cells may also be modified to alter the interaction of the target cell with the blood contacting surface.

The term “blood contacting surface” refers to a surface exposed the bloodstream of a subject which is configured to attract and recruit a targeted cell circulating in the bloodstream to the blood contacting surface. Examples of a “blood contacting surface” include but are not limited to a surface of a pre-existing medical device positioned in the bloodstream of a subject which is modified in vivo to recruit target cells, a surface of a prosthesis which when the prosthesis is implanted into the subject is exposed to the bloodstream and recruits circulating target cells, a surface exposed to a subject's circulating blood ex vivo, and also a surface of a blood vessel or artery of the subject's which is modified in vivo to recruit target cells. For exemplary purposes, the prosthesis is a stent or a vascular graft tubular in shape, comprising an exterior surface and a lumen surface in which the lumen surface is the blood contacting surface, when the stent or the vascular graft is implanted into the subject.

The term “cellularization” as used herein refers to the spreading and/or differentiation of target cells over the blood contacting surface. An example of cellularization is the formation of an endothelialized surface on the blood contacting surface when the target cell is an endothelial progenitor cell.

The term “neo-endothelium” as used herein defines a tissue structure or layer which is formed from endothelial progenitor cells or endothelial cells and resembles natural endothelium.

The term “neo-vessel” as used herein defines a permanent, bioactive, anti-thrombogenic bioartificial blood vessel or artery formed in vivo which is capable of functioning substantially like a natural blood vessel or artery.

The term “foreign body response” as used herein refers to a response from the implant recipient which causes a healing response to the graft, which leads to encapsulation of the graft or scaffolding by vascular cells to form a exterior vascular tissue structure.

The term “prosthesis” and “prosthesis surface” are used herein to refer to prosthetic devices and prosthetic surfaces made of non-biodegradable or biodegradable materials. Prosthesis blood contacting surfaces include but are not limited to a surface of stents, anastomotic devices, pacemakers, heart valve prostheses, vascular prostheses, artificial hearts, tissue engineered implants or biopolymer scaffolds such as de-cellularized heart valve grafts.

The term “vascular structure” as used herein means a highly vascular, fibrous capsule that appears around a mandrel or scaffold or support member which has substantially similar properties and functions as a natural vessel.

The term “biodegradable” as used herein refers to a material's ability to undergo breakdown or decomposition into biocompatible compounds as part of a normal biological process.

The term “bind” and its derivatives, as well as “attach,” as used herein refer to adsorption, such as, physisorption, or chemisorption, “lock and key” (ligand/receptor) interaction, covalent bonding, attraction by hydrophobic, magnetic or van der Waals interactions, hydrogen bonding, or ionic (electrostatic) bonding of a polymeric substance, peptide, protein, amino acid sequence or bioactive species to a blood contacting surface (e.g. a surface of an implanted support member or prosthesis exposed to the bloodstream).

The term “ligand” as used herein includes molecules which are binding partners to a molecule presented on the surface of the target cell. Ligands include but are not limited to enzymes, organic catalysts, ribozymes, organometallics, proteins, glycoproteins, peptides, polyamino acids, antibodies, nucleic acids, steroidal molecules, antibiotics, antimycotics, cytokines, carbohydrates, oleophobics, lipids, for example, which are capable of binding to and adhering to molecules useful in the present invention such as endothelial precursor cells (i.e. endothelial progenitor cells). In addition, non-cellular biological entities, such as viruses, and prions are considered ligands of the subject invention. Ligands may also refer to magnetic entities used to attract target cells to the blood contacting surface.

The term “immobilize,” and its derivatives, as used herein refer to the attachment of a ligand directly to a support member or to a support member through at least one intermediate component.

The term “functionalize” as used herein in connection with scaffolds means to treat or derivatize a scaffold surface to provide selected chemical functional groups thereon. “Functionalization” of a scaffold, as used herein, may involve, for example, reactive chemical treatment of a surface, or coating the surface with a layer of material providing desired functional groups, or both. Furthermore and depending on the context, functionalization is used describe the modification of a surface aimed to fulfill a certain function such as, for example, surface modification by ligand attachment to retain target cells at the prosthesis surface or the attachment of growth factors to induce the differentiation of target cells (e.g. EPCs into functional endothelial cells).

The term “coating layer” as used herein identifies a layer and or mixture which may contain specific ligand binding molecules or magnetic-particles which will be functionally attached, immobilized, bound or adsorbed onto the blood contacting surface, and may contain other molecules such as buffers, salts and polymers not found in the scaffolding support member (e.g. prosthesis) which enhance the structural integrity of the layer, buffers, salts etc.

The term “donor cell” as used herein is a cell other than the subject's cells.

Exemplary “donor cells” are cells from tissue culture, cells from a cell line, bacteria, cells from a mammal, and cells from a species other than the subjects species.

Overview

This invention pertains to devices and methods for recruiting targeted cells circulating in the bloodstream of a subject, to a blood-contacting surface in vivo. In one embodiment, the invention allows for the establishment of a permanent or temporary, bioactive, anti-thrombogenic graft by the in vivo recruitment of cells in the blood of the subject (e.g. a graft recipient) to a blood contacting surface, such as a prosthetic surface exposed to blood flow.

Recruitment of Circulating Cells to a Blood Contacting Surface In Vivo

Recruitment and targeting of cells to the blood contacting surface is accomplished by various recruitment methods such as magnetic attraction and/or molecular binding. The cells useful in the invention are cells naturally circulating in the vascular circulatory system of a subject, cells which are induced to circulate in the blood stream of a subject or cells introduced into the vascular circulatory system specifically for interacting with the blood contacting surface. Cells introduced into the vascular system may be harvested, for example, from bone marrow, fat tissue, blood and other biological tissues either from the subject or a mammalian cell donor, such as a human or a pig. The target cells introduced into the vascular system can be derived from a cultured cell line. Introduced target cells also comprise bacteria which have been genetically altered to interact with the blood contacting surface, or to express a therapeutic substance at the blood contacting surface.

Cellular Recruitment by Magnetic Interaction

In certain embodiments, magnetic attraction is utilized to recruit circulating target cells from the bloodstream to a blood contacting surface and retain these target cells at the blood contacting surface against the shear and drag forces of the blood flow. In an external magnetic field, ferro- or paramagnetic particles orient and align themselves along the field-lines of the magnetic field. While paramagnets become magnetized only in the presence of a magnetic field, ferro-magnets may already be in a magnetized state. If the magnetic field is non-homogeneous, the field gradient exerts a force on the oriented magnetic particle, attracting the particle towards the direction of higher field strength. If such a magnetic field emanates from magnetized matter, the magnetic field strength is highest at the surface of the matter. Thus a para- or ferromagnetic particle will always be attracted to the surface of magnetic matter.

In one embodiment, the target cells are magnetically modified to enable the recruitment of the modified target cells, when introduced into the subject's bloodstream, by magnetic attraction to a magnetically charged blood contacting surface. Magnetic particles may be incorporated into the cell or attached to the cell surface by procedures known to those skilled in the art. In certain embodiments, magnetic particles may be fed to the target cells (Moller W, et. Al. (1997) J Aerosol Med 10:173-186; Violante (1990) Acta Radiol Suppl 374: 153-156) or temporary pores may be created in the cell membrane of the target cell by electroporation (Moroz & Nelson (1997) Biophys J 72:2211-6; Zhelev & Needham (1993) Biochim Biophys Acta. 1147(1):89-104; Neumann E, Kakorin S, Toensing K. (1998) Faraday Discussions 111: 111-125). In other embodiments, magnetic particles may be attached to the cell surface via antibody binding to cell membrane receptors or through chemical conjugation of the magnetic particle to the cell membrane (Yin, A H; Miraglia, S; Zanjani, E D; Almeida-Porada, G; Ogawa, M; Leary, A G; Olweus, J; Kearney, J; Buck, D W (1997) Blood 90: 5002-5012; Buckley et al. ABL 1998 June 30-32).

In certain embodiments, cells may be magnetically modified or labeled by intravenous injection of magnetic particles which are conjugated to molecules which in turn will attach to the surface of the cells to be recruited to the surface. One such example constitutes the antibody-mediated binding of magnetic particles to the CD133 or CD34 protein found on the surface of several progenitor cell types. These cells may comprise endothelial progenitor cells or mature endothelial cells which may or may not have been genetically modified to express or produce an agent with an inhibitive effect on smooth muscle cell proliferation.

Para- or ferromagnetic particles may be enclosed in lipid membrane vesicles (liposomes) associated with the targeted cell or within a polymer matrix of micro- and nanoparticles attached to the cell of interest. Alternatively, the magnetic particles may be conjugated to the cellular surface of the targeted cell to constitute part of the cellular membrane. The cells are recruited from the blood stream to the magnetically charged prosthesis by magnetic attraction.

The strength of attraction depends on the magnetic properties of the particles utilized to modify the cells to be recruited to the surface, as well as the strength of the magnetic field emanating from the surface, and the gradient of this field where both the field and its gradient will vary with location. The magnetic properties of the particle depend on the chemical composition of the particle as well as its magnetization state. The properties of the magnetic field depend on surface and body geometry, the chemical composition and magnetic history of the device. Once attracted to the blood contacting surface or lumen of the prosthesis, the cells adhere to the prosthesis and in certain embodiments the cell adhesion induces cellular spreading and differentiation over the blood contacting surface of the prosthesis.

In other embodiments, increasing the targeted cell affinity to magnetized surfaces or prostheses comprises incorporating magnetic particles into targeted cells through fusion of vesicles to the targeted cell. A vesicle defines a volume enclosed by a membrane. This membrane may consist of proteins, lipids, polymers, block-copolymers, or a mixture thereof. When such a vesicle fuses with a cell, the vesicle volume becomes part of the cell plasma and the vesicle's contents are released into the cell interior. If the vesicle is loaded with magnetic particles during vesicle formation, fusion with the targeted cells results in incorporation of these magnetic particles into the cell interior.

Another technique for incorporating magnetically sensitive particles into target cells is by endocytosis. For this purpose, magnetic particles are fed to cells with endocytotic capabilities. Upon contact with a particle, cells, which may be stimulated to do so, will engulf the particle by firstly adhering their membrane to the particle, secondly increasing the area of adherence until the entire particle is enclosed by a membrane section of the cell at which time thirdly, the particle is incorporated into the cell interior by virtue of invagination of the membrane enclosed particle. In yet another embodiment, small magnetic particles with a diameter between 50 and 250 nm may be brought into the target cell by creating temporary pores in the cell membrane through electric field exposure(i.e. electroporation). These standard techniques and others useful in the methods of the invention are described by the following references, Moller W, Takenaka S, Rust M, Stahlhofen W, Heyder J. (1997); J Aerosol Med 10:173-186; Violante (1990) Acta Radiol Suppl 374: 153-156); Moroz & Nelson (1997) Biophys J. 72:2211-6; Zhelev & Needham (1993) Biochim Biophys Acta. 1147(1):89-104.

Attachment of Magnetic Particles to Cell Membrane

Modification of cell magnetic properties in certain embodiments, comprises attaching magnetically sensitive particles, such as ferromagnetic or paramagnetic particles including but not limited to ferrite, samarium cobalt, or neodymium boron particles to the surface of the targeted cells. This may be achieved by modifying the surface of these particles to have affinity for the membrane of the targeted cell. This affinity may be established by attaching ligand molecules (binding partners) to their appropriate cell surface molecule found on the targeted cell membrane, e.g. antibodies to a cell surface receptor, to the surface of the particle. The binding of magnetic particles to the cell membrane may also be achieved by reacting the magnetic particle, or a particle-encapsulating polymer matrix, to molecular groups typically found at cell membranes, including such groups as amine or thiol or hydroxyl groups, through chemically reactive groups presented at the particle or matrix surface.

In other embodiments, the target cell can be modified to be magnetically charged by encapsulating the magnetic particle within or attached onto a polymeric matrix that is modified to have an affinity to the target cell membrane. The surface of magnetic particle and/or the polymeric matrix may comprise proteins or peptide sequences, e.g. such as RGD peptides, which provide sites of attachment for target cell surface integrins.

Standard protocols as described by Kemshead J T, Ugelstad J. (1985) Mol Cell Biochem 67: 11-18, have been utilized to magnetically modify target cells of the invention. The size of these particles is dependent on target cell type as well as the desired strength of the magnetic attraction. The magnetic particles useful in the invention have a diameter which ranges from about 50 nm to about 5 μm, more typically iron about 100 nm to about 1 μm.

Cellular Recruitment via Ligand Interaction

In certain embodiments, specific interactions between target cell surface molecules such as receptors, and ligands or antibodies can be used to recruit circulating target cells from the blood stream to a blood contacting surface and also retain these cells on the surface against the shear and drag forces of the blood flow. The present invention provides a ligand (e.g. an antibody to a specific cell surface receptor) on the blood contacting surface (e.g. a blood contacting surface (lumen) of a prosthesis, surface of a medical device such as a heart valve or a cellular denuded surface of a natural blood vessel) which is specific for a receptor or other surface molecules such as polysaccharides, integrins, or previously introduced lipid-anchored peptides associated with the circulating targeted cell. The probability of cellular recruitment to the surface depends on the probability of the establishment of a target cell surface molecule-ligand interaction or bond, the strength of a single bond, the surface concentrations of the specific cell surface molecules and concentration of the ligands on the blood contacting surface, the concentration of the targeted cells in the blood stream and the force exerted on the target cells by the blood flow. In certain embodiments, the cell surface molecule targeted by the ligand conjugated on the blood contacting surface may be a naturally expressed receptor specific for a particular cell type or group of similar cells such as the CD34 or CD133 receptor or KDR expressed on circulating progenitor cells cell surface, CD4 receptor found on T helper cells cell surface, P-selectin and/or CD140 which are expressed on platelet surfaces. Polysaccharides, glycoproteins and glycophorin are also useful as cell surface molecules targeted by ligands presented on the blood contacting surface for attracting target cells such as erythrocytes. The ligand and targeted cell surface molecule can be defined as binding partners. Exemplary binding partners (ligand to targeted cell surface molecule) useful in the recruitment of a target cell to a blood contacting surface include but are not limited to antibodies:CD receptors, VEGF:KDR, serum specific antibodies: glycophorin; integrins as ligand to extracellular matrix proteins (e.g. RGD amino acid sequence).

In other embodiments, modification of the target cell in vitro involves presenting a novel molecule, such as an antibody onto the cell surface of the target cell: The presented molecule specifically binds/interacts with the ligand on the blood contacting surface, thus increasing the interaction between the target cell and the blood contacting surface. Modifying the targeted cells may comprise anchoring the specific molecule (e.g. receptors/antibodies) into the cell membrane by conjugation to a lipid or a transmembrane protein, or the receptor maybe genetically or chemically engineered to comprise a transmembrane protein domain for insertion into the cellular membrane of the targeted cell as described by Guan J L, Rose J K. (1984) Cell 37: 779-87.

In other embodiments, the selected cell surface molecule is chemically conjugated or physically adsorbed to the cellular surface or to a carrier agent bound to the targeted cell. Representative protocols for chemically conjugating or physically adsorbing a molecule to a cell surface are found in the following reference, Ludwig, F. (1999) Dynamic Strength of Molecular Anchoring and Material Cohesion in Fluid Biomembranes, PhD Thesis, Technical University of Munich, Germany.

Cell surface modification may comprise attachment of a molecule (e.g. a peptide or protein such as a receptor) to the cell surface which facilitates cell attachment to one or more binding partners (e.g. ligand) on the blood contacting surface or initiates target cells spreading or differentiation on the blood contacting surface. The cell surface molecule (e.g. modifying agent) utilized in cell surface modification maybe a peptide, protein, polysaccharide, antibody, receptor, a polymer or a combination thereof The cell surface molecule can be a unifunctional, bifunctional or multifunctional cell modifying agent. Multifunctional agents, in certain embodiments, comprise at least two different domains which allow the binding of two distinct ligands presented on a blood contacting surface.

In other embodiments, the targeted cells may be stimulated (either in vitro or through an agent injection in vivo) to express specific molecules such as receptors at their surface which interact with the ligands presented on the blood contacting surface, or a therapeutic substance which may be released from the cell to tissue in the vicinity of the blood contacting surface. Modification of the cells in vivo can occur either pre or post attachment to the blood contacting surface. Compounds which alter the characteristics of the target cell, i.e. compounds which induce retention, spreading or cell differentiation may be injected into the subject, or may be released from the blood contacting surface, or may be attached to the blood contacting surface, to modify the target cell. An example of target cell modification in vivo is the injection or surface release of cytokines and growth factors such as VEGF121, VEGF165, VEGF189, bFGF, aFGF, P1GF, PDGF and the like, into the subject to induce spreading and differentiation of endothelial progenitor cells.

In one embodiment, the target cells are genetically transfected to express specific molecules such as receptors at their surface which interact with the ligands presented on the blood contacting surface. After cells have been transfected with a gene with a generic promoter and a sequence encoding a specific surface receptor, the cells transcription machinery will translate the genetic code into the particular surface protein which thereafter is displayed on the cell's surface.

Mobilization of Target Cells

Compounds known to mobilize target cells, and enhance the concentration of target cells in the blood stream include, but are not limited to vascular endothelial growth factor (VEGF), stem cell factor (SCF), granulocyte-macrophage colony-stimulating factor (G-CSF) or granulocyte colony-stimulating factor (G-CSF). These target cell mobilization compounds may be administered to the implant recipient (patient) in an effective amount and/or presented on the endothelializable surface of the blood contacting surface to enhance the level of target cells in the recipient's blood stream. VEGF, GM-CSF and G-CSF can be introduced into the circulatory system by intravascular injection (e.g. 100 micrograms per day), Asahara T, et. al., EMBO J. 1999 July 15;18(14):3964-72. Mobilization of the target cell may also be enhanced by increasing the serum levels of VEGF, GM-CSF and G-CSF (Kalka C, et. al. Ann Thorac Surg. 2000 September; 70 (3):829-34). It may also be desirable to augment the number of circulating target cells by systemic stimulation and/or by exercise in order to support the (in vivo) cellularization process.

In certain embodiments, progenitor cells are mobilized from their respective resident tissues (such as for example the bone marrow) by virus mediated cell transfection in vivo. This method provides a sustained release of the mobilizing compound over time, which in turn results in a sustained increased blood concentration of circulating progenitor cells.

Target Cell Modification

In one embodiment, target cell surface modification comprises, fusing a vesicle with the targeted cell. A vesicle defines a volume enclosed by a membrane. This membrane may consist of proteins, lipids, polymers, block-copolymers, or a mixture thereof. When such a vesicle fuses with a cell, the vesicle membrane becomes part of the cell membrane, thus introducing the molecular compounds of the vesicle membrane into the cell membrane. In this embodiment, this is used to incorporate specific receptors into the cell membrane of the target cell, to enhance the binding affinity for the blood contacting surface. Such receptors include but are not limited to transmembrane proteins, lipid-conjugated peptides or proteins or polysaccharides. The receptors incorporated by vesicle membrane fusion may increase cell affinity to the blood contacting surface, but may also act as signal transducers, increasing the cell's sensitivity to external stimuli such as chemical, mechanical or electrical stimuli can initiate a signaling cascade which results in a cell surface or cytoskeleton modification.

In other embodiments, increasing the affinity of targeted cells to the blood contacting surface comprises introducing a ligand binding partner onto the target cell surface by attaching the ligand binding partner to a cell surface protein accessible on the target cell. Thus modifying existing membrane proteins into targeted cell surface molecules recognized by the ligands on the blood contacting surface. For exemplary purposes, if avidin is the ligand on the blood contacting surface, biotin can be introduced onto the cell surface of the targeted cells by attaching biotin to pre-existing cell surface proteins. This may be achieved by attaching a biotinylated antibody, the antibody specific for a certain cell membrane protein, resulting in the display of biotin molecules at the target cell surface.

In an alternative approach, the cells signaling pathways may be targeted to increase the expression of specific surface receptors or stimulate the expression of a therapeutic substance once the cell has been recruited to the surface. This signaling may be achieved by binding of solutes to specific cell receptors.

In another in vitro approach to increase the affinity of targeted cells to the immobilized ligands on a blood contacting surface, binding partners to the ligands are chemically conjugated to the cell membrane. Chemical conjugation can be achieved by reacting these binding partners to components of the cell membrane through crosslinking agents, photochemically sensitive groups, reactive esters, cyano acrylates, maleimide groups, epoxy groups, or other such chemistry as known to those skilled in the art.

In certain embodiments target cells are genetically altered to increase affinity to the blood contacting surface by promoting the expression of particular membrane proteins on the surface of the target cell or to promote the expression of a therapeutic substance after the target cell has been recruited to the blood contacting surface. In one aspect of the invention, genetic sequences for cell surface molecules are incorporated into an appropriate vesicle, and introduced into the cell by vesicle fusion. A vesicle defines a volume enclosed by a membrane, the membrane being synthetic or naturally occurring. The membrane may consist of proteins, lipids, polymers, block-copolymers, or a mixture thereof. When such a vesicle fuses with a cell, the vesicle volume becomes part of the cell plasma and the vesicle's contents are released into the cell interior. If the vesicle is loaded with a gene sequence during vesicle formation, fusion with the targeted cells results in incorporation of the sequence into the cell interior. After successful transfection, the cell expresses the molecule encoded by the gene sequence. When the encoded sequence is for a cell receptor (e.g. CD34 or CD133) on the cell surface of the target cell, which is the binding partner for the ligand on the blood contacting surface, the densitiy of this receptor is increased, providing enhanced ligand (binding partner)receptor interaction. Standard protocols for gene delivery to a host cell are described in the following references, Tari A M, Tucker S D, Deisseroth A, Lopez-Berestein G. (1994) Blood 84:601-7, U.S. Pat. Nos. 6,110,490 & 5,908,635 & 5,624,820 & 5,976,567; Nahde T, Muller K, Fahr A, MullerR, Brusselbach S. (2001) J Gene Med 3:353-61).

In other embodiments, electroporation is utilized to transfect target cells. In general, electorporation the cells are temporarily subjected to electric fields in order to create transient pores within the cell membrane. During pore formation, a sequence of DNA or RNA encoding a cell surface molecule is present in the vicinity of the cell, allowing the sequences to diffuse through the pores into the cell interior. To increase transfection efficiency, the electrical field may be repeatedly applied in a pulsed mode. Concentration of genetic sequences, electric field strength, pulse duration and the number of pulses may have an effect on transfection efficiency.

In other embodiments, vectors such as a viruses are used to genetically transfect the targeted cells in order to either to increase affinity to the target surface by promoting the expression of particular membrane proteins, or to promote expression of a therapeutic substance once the cell has been recruited to the blood contacting surface, In this embodiment, part of the viruses genetic code is altered or amended by the genetic sequence which is to be delivered to the cell. This method utilizes the viruses natural infection capabilities to deliver a genetic payload. The protein sequences encoded by this payload will be expressed by the cell once the delivery has been completed, or, depending on the promoter sequences conjugated to the genetic sequence, upon binding of particular transcription factors to the specific promoter. The binding of the transcription factors may in turn be regulated by external events or intracellular signaling pathways, in this way making the expression of the delivered gene dependent on the occurrence of a particular event. The use of viruses as a gene delivery vehicle allows the specificity of certain viruses to infect particular cell types, thus enabling gene delivery to target cells both in vitro and in vivo.

Cellularization of a Blood Contacting Surface by Target Cells

The target cells of the present invention include cells which bind, spread and differentiate across the blood contacting surface to form a stable and long lasting cellular covering over the blood contacting surface. Other target cells of the invention such as, platelets, macrophages and cells of the immune system such as T helper cells interact temporarily with the blood contacting surface to alter specific characteristics of the blood contacting surface or other blood circulating molecules. In certain embodiments multiple cell types are targeted by the blood contacting surface to allow both the cellularization of the blood contacting surface as well as modify the blood contacting surface temporarily or modify the cells adhering to the blood contacting surface.

Endothelialization of a Blood Contacting Surface

Endothelialization of synthetic grafts has proved a substantial challenge to the use of small diameter grafts in cardiovascular surgery. The present invention delineates an approach which results in the in vivo endothelialization of a synthetic graft by recruiting endothelial progenitor cells to the graft blood contacting (lumen) surface. This invention also pertains to devices and methods for developing endothelialized structures in vivo. The invention allows for the establishment of a permanent or temporary, bioactive, anti-thrombogenic graft by the in vivo recruitment of endothelial progenitor cells (EPC) in the blood of a graft recipient to a prosthetic surface exposed to blood flow, followed by the subsequent retention, spreading and differentiation of the adhered endothelial progenitor cells to allow the formation of a functioning endothelium. In certain embodiments, vascular tissue forms around and encapsulates the non-blood contacting surfaces of the prosthesis, simultaneously with the formation of the viable neo-endothelium on the blood contacting surface.

Exemplary surfaces for endothelialization on prostheses other than vascular conduits include but are not limited to tissue engineered or synthetic implants such as heart valves, artificial hearts, or tissue engineered or synthetic organs such as liver tissue, heart muscle patches, or bone or cartilage scaffolds. While some of these prostheses are still in the experimental stage, others, such as heart valves and artificial hearts are clinically available at present.

EPC Recruitment Retention and Spreading

The recruitment of EPCs to the endothelializable surface of the prosthesis is facilitated by modifying or functionalizing the blood contacting surface selected to be endothelialized. The selected surface of the prosthesis may be functionalized with ligands that bind and retain EPCs to the prosthetic device with high specificity or magnetically charged. The surface of interest can be modified to present ligands which specifically recognize EPC surface molecules such as CD34 receptor, CD133, KDR (VEGFR-2), VE-Cadherin, E-selectin, αᵥβ₃ EPC specific lectins, or other EPC surface molecules. EPC ligands may, for example, comprise antibodies, antibody fragments, proteins, peptides, nucleic acids, antibodies or any other molecule which substantially binds only endothelial precursor cells such as EPCs.

To retain EPCs and allow EPCs to spread onto the endothelializable surface, focal adhesion receptors found on the EPCs are presented to the peptides/ligands immobilized on the endothelializable surface of the prosthesis. These peptides/ligands provide an attachment point for the cells and promote cell functionality and cell retention at the surface. Endothelial progenitor cell spreading may proceed, follow, or occur simultaneously with differentiation to endothelial cells. EPC attachment with the ligands presented on the lumen surface, may also induce cell differentiation. Molecules specific for cellular spreading may also be coated or attached to the lumen surface as a mixture with EPC binding ligands or added alone. Examples of molecules which enhance cellular spreading include, by way of example, peptides with the amino acid sequence RGD or REDV, fibrin, fibronectin, laminin, gelatin, collagen, basement membrane proteins and the like. The spreading molecules are present on the endothelializable surface to optimize cell spreading without interfering with the EPC binding specificity to the surface.

In certain embodiments, EPCs are recruited and retained by utilizing an EPC marker compound. In this embodiment, the recipient is administered an “EPC marker” compound which specifically binds to EPC's, and the endothelializable surface of the prosthesis is modified to bind to the EPC marker compound. The EPC marker can be modified to allow binding to the endothelializable surface by covalent and/or hydrogen bonding as well as magnetic or electrostatic (ionic) forces.

For enhancing the retention and spreading specificity of EPCs to the selected surface of the prosthesis, the surface may be coated with a layer of shielding molecules, for example hydrophilic polymers, to decrease the nonspecific binding of cells other than the desired endothelial precursor cells. These shielding molecules may be mixed with a coating layer comprising the EPC binding ligands or as a separate layer. The process of binding EPC ligands to the functional groups on the scaffolding may be incomplete and from about 10 to 80% of the functional groups on the scaffolding may remain unattached to an EPC ligand. Shielding molecules may block these exposed functional groups and shield or block them from binding to non-specific and unwanted molecules such as platelets or cells other than EPCs.

EPC Differentiation

In order to cover the endothelializable surface with a confluent cell lining substantially similar to that found in natural vessels, the surface-bound progenitor cells need to differentiate into endothelial cells. This differentiation may be induced by inherent mechanical stimuli such as shear stress or naturally available biochemical stimuli from the circulation (e.g., plasma proteins, tissue bound growth factors, growth factor stabilizing agents such as hepartin, etc). Cell differentiation may precede, occur during or follow cell spreading. Molecules involved in EPC differentiation which may be presented on the endothelializable prosthetic surface include but are not limited to, VEGF, FGF and SCF. In one embodiment the endothelializable surface of the scaffolding comprises EPC ligands as well as molecules which stimulate and promote the differentiation of adhered EPCs into endothelial cells. These differentiation promoting molecules may be unique or substantially similar to the ligands promoting initial adhesion and/or cell spreading of EPCs.

The time necessary to accomplish endothelial coverage of the endothelializable surface of the scaffolding is dependent on the blood concentration of circulating progenitor cells, the size and shape of the surface to be endothelialized, affinity between ligand and EPC surface molecules, the concentration of EPC ligands on the scaffolding, the concentration of EPC surface molecules targeted by the EPC ligands on the scaffolding, force resistance and shear stress of the blood flow path as well as other factors.

Blood Contacting Surfaces

In the present invention, blood contacting surfaces comprise surfaces of a preexisting medical device positioned in the bloodstream of a subject which is in contact with circulating blood. A blood contacting surface of a pre-existing medical device is modified in vivo by coating the blood contacting surface of the medical device with a coating comprising ligands or magnetic particles which attract and recruit target cells. In other embodiments, a surface of a prosthesis which when the prosthesis is implanted into the subject, is exposed to the bloodstream is considered a blood contacting surface. In this embodiment, the prosthesis is prepared prior to implanting into the patient so that the blood contacting surface comprises a ligand specific for a target cell and/or is magnetically charged to attract modified target cells. In other embodiments, the blood contacting surface is exposed to a subjects circulating blood and molecules, including target cells circulating in the blood ex vivo. In an ex vivo embodiment, the blood contacting surface is positioned within the blood stream of the subject, but outside to subject's body.

In other embodiments, the blood contacting surface is a blood vessel or artery of the subjects which has been striped of its natural cells by medical procedures or by natural causes. The blood contacting surface in this instance may be coating in vivo with a coating that attracts specific target cells, such as endothelial progenitor cells to cellularize the injured blood vessel or artery. The coating may comprise ligands specific for cell surface molecules of the target cell or enable the magnetic attraction of magnetically modified target cells circulating in the blood stream of the subject.

Blood Contacting Surface Modification

In certain embodiments, the blood contacting surface of the implant exposed to the circulatory system may comprise compounds which attract the cell of interest by magnetic or electrostatically charged forces. The prosthetic structure comprises magnetic particles configured so as to attract specific cells or blood components which have been modified to be magnetically charged. Compounds within the surface of the implant (scaffolding) maybe modified to comprise a magnetic component, and the agent (e.g. cell) of interest maybe modified to also comprise a magnetic component to target the agent magnetically to the surface of the scaffolding.

In other embodiments, the blood contacting surface comprises at least one ligand specific for the targeted cell. The ligand on the blood surface may comprise a peptide, protein, polysaccharide, or specific chemical substrate having a moiety specific for a particular molecule presented on the surface of the targeted cell. In some embodiments, a plurality of various specific ligands is present on the blood contacting surface, allowing the interaction with multiple types of cell surface molecules (e.g. receptors) on the surface of a targeted cell.

In other embodiments, proteins such as growth factors capable of stimulating cell mobilization, cell proliferation or cell differentiation may be loaded into the polymer matrix which coats the blood contacting surface. Exemplary polymer matrices include glutaraldehyde crosslinked gelatin matrices, calcium alginate hydrogels or chitosan hydrogels. Crosslinked gelatin matrices for example may be fabricated by mixing a 12% gelatin in water solution with a 1-5% solution of glutaraldehyde. Calcium alginate gels may be made by introducing a 1.2%-1.5% sodium alginate solution into an excess of 80-120 nM calcium chloride. The gels may be soaked during or after formation in phosphate buffered saline solution containing the protein (e.g. growth factors) to be released at a concentration of about 10 nM to about 100 nM. In certain embodiments it is useful to include ligands such as heparin for VEGF, FGF or a carrier protein such as serum albumin into the gel, in order to stabilize the active protein against degradation (Lo H, et al. J Biomed Mater Res 1996 April; 30(4):475-84; Lopez J J, et. al. Am J Physiol 1998 March; 274(3 Pt 2):H930-6; Cleland J L, et. al. J Control Release. 2001 May 14; 72(13):13-24; Tabata Y, et. al. J Biomater Sci Polym Ed 1999;10(1):79-94).

In certain embodiments, heparin is chemically conjugated to the surface. Subsequently, the surface is incubated in a phosphate buffered saline solution containing a defined amount of VEGF or FGF, usually on the order of about 1 to about 1000 nM for approximately one hour at room temperature. During this step, the heparin binding growth factor, e.g. VEGF or FGF is allowed to bind to the surface-immobilized heparin. The surface may then be lyophilized and stored. Upon implantation, the growth factor dissociates from its heparin bond over time, thus providing a sustained release of the growth factor over time (Edelman E R, et. al., Biomaterials. 1991 September; 12(7):619-26).

In one embodiment of the invention, the blood contacting surface is not part of a prosthetic implant, but is constructed in situ to recruit circulating target cells from the blood stream. The surface may be constructed to comprise at least one ligand, e.g. a compound which binds surface receptors of the target cells, or it may be constructed to attract target cells via magnetic interaction. This embodiment of the invention is particularly useful to attract progenitor cells, e.g. endothelial progenitor cells, to sites of endothelial denudation or vascular injury.

In one method used to construct a blood contacting surface in situ, biological glue, e.g. fibrin glue, is delivered to the target site via a catheter. The glue may be delivered to the target site through pores in the distal end of the catheter, or through 100-200 nm diameter pores in an inflatable balloon. In order to construct a blood contacting surface which attracts target cells by molecular interaction, ligands to target cell surface receptors, e.g. CD34 or CD133 antibodies, are conjugated to the glue's compounds, e.g. to fibrinogen molecules, before gelling the glue in situ. The conjugation may be achieved by amine reactive esters, by epoxy chemistry, by photochemically sensitive crosslinking agents or by multifunctional crosslinkers. For fibrin glue gelling within 20 to 30 seconds, typically 2.5 mg/ml fibrinogen are mixed with thrombin of 0.1 NIHU/ml. Gelling times can be adjusted by the thrombin concentration (Kipshidze et al (2000) Journal of the American College of Cardiology 36: 1396-1403).

In one method used to construct a surface which is capable of attracting target cells via magnetic interaction, magnetized microspheres made from samarium cobalt or from neodymium boride and of a diameter in the range of 10-50 microns are mixed into the fibrin glue. When magnetically labeled target cells circulate within the vicinity of this surface, they are attracted to this surface through magnetic attraction. Target cells may be magnetically labeled by any one of such methods as endocytosis or phagocytosis of magnetic particles, vesicle fusion or attachment of magnetic particles to the cell surface via molecular binding, as outlined elsewhere in this invention.

Introducing Ligands to a Blood Contacting Surface

The prosthesis/scaffolding of the present invention comprises at least one functionalized blood contacting surface to allow for the attachment of target cell binding ligands, as well as other molecules such as, target cell mobilization enhancer molecules, molecules for cellular retention and spreading, molecules for target cell differentiation as well as possible pharmaceutical compounds. The functionalization of a surface of the prosthesis may involve gas plasma treatment, chemical modification, photochemical modification, chemical modification through y-radiation activation, co-polymerization with molecules containing functional groups, as well as other surface modification techniques well known in the art. The surface to be modified may, for example, be subject to ozonolysis to introduce carbonyl or other reactive groups thereon which will facilitate the attachment of ligands of interest to the chosen blood contacting surface. The blood contacting surface may be subject to treatment with acid or base solutions to form hydroxyl and/or carboxylic acid functionalities thereon. Functionalization of the surface to be modified may also be achieved by coating the blood contacting surface or lumen surface of the prosthesis with a layer of polymeric material having a desired functionality. Such polymer layers may comprise, for example, polyamines such as poly(L-lysine) and poly(L-glutamine) to provide amine functionalities on the specified surface.

In one embodiment, a first layer is coated onto a surface of the prosthesis comprising molecules with functional groups, the functional groups including but not limited to primary and secondary amine groups, carboxyl groups, sulfhydryl groups, and hydroxyl groups. The first layer comprising the above mentioned functional groups allows for the further attachment of target cell binding ligands and other molecules useful in retention, spreading and cell differentiation, which may be added in additional coating layers on the specified surface of the prosthesis. In other embodiments, the functionalized groups, target cell binding ligands, and molecules used to enhance retention, spreading and differentiation may be comprised in a single surface coating. In some embodiments, it may also be desirable to add biofunctional molecules to one surface coating layer while others may be added in additional layers. In some embodiments, it may be useful to introduce a linker/spacer molecule between the target cell binding ligands and other molecules useful in retention, spreading and cell differentiation. It may be desirable to conjugate the biofunctional molecule (e.g. EPC binding ligands and other molecules useful in retention, spreading and cell differentiation) to the linker/spacer first and to attach this conjugate to the surface, or, it may be desirable to attach the linker/spacer molecule to the surface first and to conjugate the biofunctional molecule to the free end of the linker/spacer molecule in a subsequent step.

It may also be desirable to introduce a homogeneously distributed mixture of functional groups in the first coating layer on the surface to be modified at defined ratios in order to make the surface multifunctional. This functionalization of the surface of the prosthesis allows for further surface modification. For example, allowing the attachment of molecules responsible for the recruitment of the progenitor cells to amine groups while attaching molecules responsible for cell differentiation onto carboxyl groups. Alternatively, the surface may be made multifunctional by conjugating a defined ratio of biofunctional molecules to one type of functional group. It may also be desirable to use a combination of the above to design a multifunctional surface.

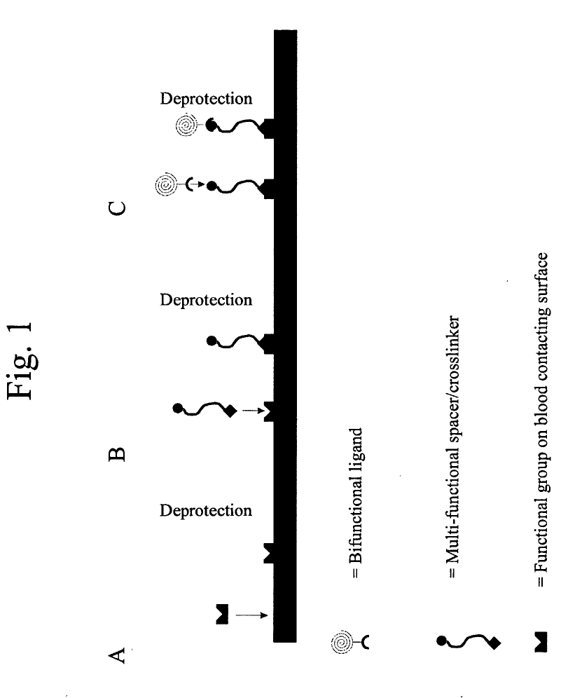

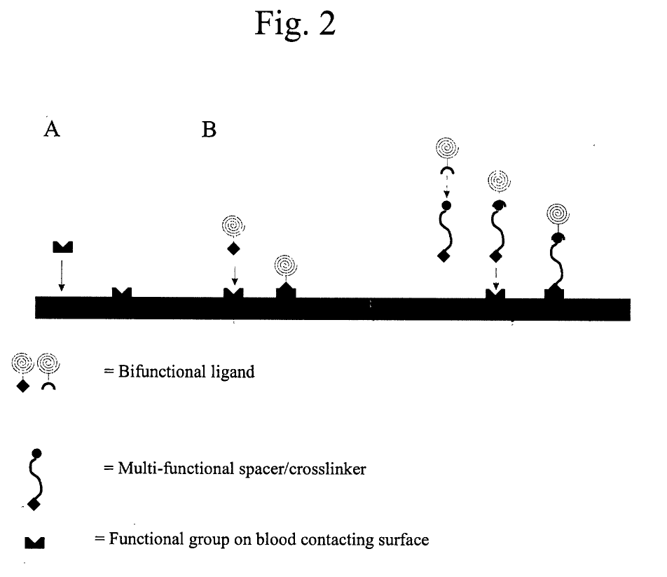

In the present invention, surface biofunctionalization may be achieved by conjugating specific molecules/peptides such as target cell ligands and other molecules useful in target cell retention, spreading and differentiation, or a conjugate of one such molecule with a linker/spacer molecule onto the functional groups available at or introduced onto a surface of the prosthesis or to the free end of a linker/spacer molecule which had been attached to the functional group in a prior step. FIG. 1 and FIG. 2 illustrate methods for surface functionalization in accordance with the invention.

FIG. 1 is a schematic of an exemplary modification procedure for the functionalization of the blood contacting surface. FIG. 1, section A, depicts the introduction of functional groups onto the blood contacting surface. These functional groups are targeted in subsequent modification steps and to immobilize biofunctional molecules and/or multi-functional spacers providing attachment sites for these biofunctional molecules. In some circumstances it may be desirable to introduce protected functional groups. These will be made accessible for further modification by a deprotection step. FIG. 1, section B, depicts multifunctional spacers/cross-linkers conjugated to the functional groups present on the blood contacting surface. As a schematic example, hetero-bifunctional cross-linking polymer chains are shown here. Again, it may be convenient to protect one moiety of the bi-functional linker during the surface conjugation and to unprotect this moiety in a subsequent step. FIG. 1, section C, demonstrates surface immobilization of the biofunctional molecules by reaction or binding to the free moiety of the cross-linker. Comprising the last and most exposed modification layer, the immobilized biofunctional molecules will determine surface properties and functionality. Again, it may be convenient to protect molecule during the conjugation step and to deprotect this molecule in a subsequent step.

FIGS. 2A-B is a schematic of another embodiment of the procedures utilized in the functionalization of the blood contacting surface. FIG. 2, section A, shows the surface modification procedures shown in FIG. 1, section A, functional groups are introduced onto the surface. In lieu of conjugating a cross-linking molecule to the surface first, the (possibly modified) biofunctional molecule is either reacted to the functional group directly or it is reacted to the cross-linker prior to the cross-linker's conjugation to the surface, FIG. 2, section B.

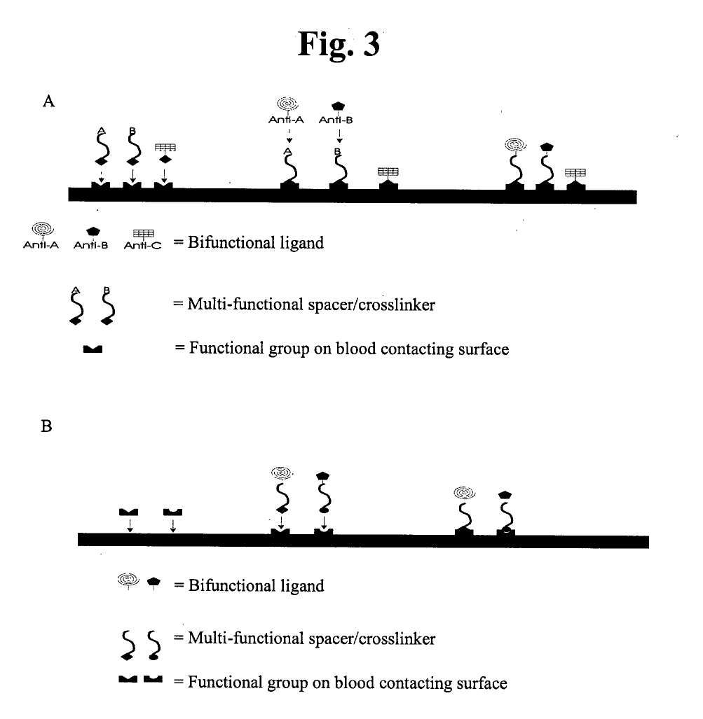

FIG. 3 illustrates a multifunctional surface design in accordance with the invention. FIG. 3, section A illustrates a multi-biofunctional surface may be constructed by surface-conjugating different spacers/crosslinkers specific for one particular biofuctionality at well-defined ratios and or by simultaneously conjugating biofunctionalities directly to the surface. Different spacers/crosslinkers may have different lengths in order to alter the accessibility to the biofunctionalities. FIG. 3, section B depicts a multi-bifunctional surface which may also be constructed by introducing a well-defined mixture of functional groups onto the surface.

Specific molecules may also be mounted onto homo or hetero multifunctional spacers (such as hydrophilic polymer chains) to extend their reach from the surface and to increase their sampling volume. For example, coupling of the desired molecules may be achieved through bifunctional cross-linkers and/or cross-linking polymers, one end of which reacts with a functional group on the surface of the prosthesis or a surface coating layer, while the other functional group of the bifunctional spacer reacts with or binds to the desired molecule to be presented on the modified surface of the prosthesis. These reaction/binding steps may be performed as separate modification steps or in combination.

Examples for coupling reaction to functional surface groups include, but are not limited to, (a) coupling to primary amine groups can be achieved by reactive esters or epoxy groups; (b) coupling to secondary amine groups can be achieved through reaction with ketone or aldehyde groups; (c) coupling to carboxyl groups can be achieved by activation of the carboxyl groups (e.g., by EDC, Pierce) and subsequent reaction with amine groups; (d) sulfhydryl groups can be targeted by a variety of reactive moieties such as maleimides and vinylsulfones; (e) coupling to epoxy groups can be achieved by primary amines or hydroxyl groups; and (f) coupling to hydroxyl groups can be achieved through silanization or through expoxide groups. Available silanes include: ammo-silanes, epoxy-silanes, sulfhydryl-silanes. The amino groups and sulfhydryl groups can be targeted in further modification steps as suggested above in (a), (d) and (e).

In one embodiment, the presentation of specific molecules/peptides could be achieved by avidin-biotin bridging: avidin or one of its related forms (e.g., streptavidin) may be immobilized at the surface via direct conjugation to a functional group on the prosthesis surface or in a first coating layer, or by binding to a biotin conjugated to a functional group in a prior preparation stage. After the immobilization of avidin onto the lumen, a biotinylated derivative of the desired molecules to be presented (e.g., EPC ligands) can then be bound to the outer layer of the surface by attachment to the avidin molecules. In analogy to the binding partners avidin and biotin, any other receptor-ligand pair of sufficient specificity and affinity may be used. Alternatively, a photo-reactive group can be used to couple the desired molecules to the surface. This also applied to coupling biotin or avidin or one of its related forms (e.g., streptavidin) to the surface.

Prosthesis Structures

In the present invention, the prosthetic structure may be a graft or implant which has at least one surface that is in contact with the subjects vascular system. Exemplary implants are a stent, an anastomotic device, a diagnostic device, a pacemaker, a heart valve, a vascular graft, a synthetic organ, an artificial heart, a prosthesis, a drug delivering pump, a graft, an autologous graft, a homograft, a xenograft, or a tissue engineered graft. In certain embodiments, the graft may be a blood vessel graft, an organ graft, a heart graft, a lung graft or a kidney graft.

The blood contacting surface of the prosthesis or medical device is configured to recruit the desired cells. The prosthesis maybe a stent, implant, heart valve or any other structure or scaffolding in which at least one surface is exposed to the vascular circulatory system. The surface of the scaffoding/implant which is exposed to the vascular circulatory system maybe coated with one or more layers to enhance the attraction of the cell of interest to the blood contacting surface of the implant. The coating maybe composed of bio-polymers, synthetic polymers and the like. The coating may further comprise a compound (e.g. ligand or substrate for a receptor or antibody) which specifically interacts with the cell of interest. The ligand may be conjugated to the coating or absorbed into the coating by techniques utilized by those skilled in the art of producing functional coatings for medical devices. The ligand in the coating may be crosslinked to an interior surface or coating of the implant. The surface may be plasma treated to introduce functional chemical groups to the implant surface to allow the ligand to covelantly attach to the surface of the implant. The functional groups useful for binding to the compound of interest consist of one or more of amine groups, carboxyl groups, hydroxyl groups, aldehyde groups, epoxy groups, acrylates.

In other embodiments, the cells are recruited to the blood contacting surface by magnetic means as described above. This approach necessitates that the implant be made from magnetic material and be in a magnetized state upon implantation or magnetizable through external magnetic fields post-implantation. Cells modified by magnetically sensitive particles are attracted to the implant surface when circulating in the vicinity of that surface.

In certain embodiments, a coating of the blood contacting surface of the prosthesis allows substances to be release over a period of time, which mediate the chemotactic attraction of the targeted cells over a period of days or weeks, or which induce the targeted cells to increase expression of specific cell surface receptors which are binding partners to ligands immobilized on the implant surface. The period of time in which the compound (e.g. ligand) may be released from the time release coating ranges from about 1 day to about 90 days. The compound in the coating may comprise peptides, proteins or synthetic compounds.

In other embodiments, the coating may comprise compound or ligands specific for the recruitment, adhesion, differentiation, proliferation spreading of the targeted cells or a combination thereof on the blood contacting surface of the prosthesis. In particular, the coating may comprise cytokines, growth factors, for example, VEGF or FGF, or substances which mimic the molecular structure of cytokines or growth factors, such as partial sequences or mutations of the binding site of growth factors or synthetic polymer casts which reproduce the molecular geometry of a cytokine, to initiate a particular cellular response aimed at stabilizing the surface population by the cells through cell spreading, differentiation, proliferation or activation.

Scaffolding Configuration

In certain embodiments, the scaffolding for a prosthesis comprises a support member which has a tubular shape configured to function as an artificial blood vessel, the scaffolding having an inner blood contacting surface (lumen) and an outer surface. The scaffolding will typically have a tubular, cylindrical configuration and is matched in size and shape to a blood vessel of interest. The support member has at least two openings which are capable of being fluidly connected to one or more blood vessels in such a manner as to allow blood flow through the openings and within the lumen (blood contacting surface) of the support member. Thus, the diameters of the openings of the scaffolding are substantially similar to the diameter of the blood vessel(s) or other vessel sections to which the scaffolding is attached. The diameter of the scaffolding openings range from about 1 to 8 mm, and typically range from about 2 to 6 mm and more typically range from about 2 to 4 mm. The length of the scaffolding depends on the extent of diffusivity of plaque or stenosis within the blood vessel being bypassed. In general, the length of the scaffolding may range from about 1 to 40 cm, more typically from about 10 to 20 cm. In one embodiment, the scaffolding comprises biodegradable materials and/or layers which promote the generation of a neo-vessel of the present invention.

Prosthesis Materials

Non-Biodegradable Prostheses

A non-biodegradable prosthesis useful in the present invention is typically of a metal such as for example stainless steel, nitinol, titanium, gold, silicone, superelastic alloys and other metals or a suitable polymeric plastic such as, for example, polytetrafluoroethylene, polyethylene terephthalate or a plastic from the family of polyurethanes, polyesters or polyethylenes. In general, the non-biodegradable prosthesis materials are constructed out of stainless steel, titanium or any other material which is suitable of implantation into a body passageway. It is to be understood that any intravascular prostheses or medical devices which are implanted into the intravascular lumen (i.e. bloodstream) of recipients may be layered with a target cell binding ligand coating of the invention. Examples of such intravascular prosthesis include, but are not limited to, those of U.S. Pat. Nos. 4,733,665 and 5,102,417.

The surface of a prosthesis made of non-biodegradable materials can be activated to allow for target cell ligands to be attached to the surface of the prosthesis. Activation of the prosthetic surface can be achieved by many methods known to those skilled in the art. These methods include but are not limited to gold plating (as gold is known to bind thiol groups), gas plasma treatment, chemical etching, covalent or ionic bonding of molecular moieties by chemical reaction or by photoreactive chemistry. Activation of a non-biodegradable prosthesis may comprise a surface coating to provide functional groups which can bind to ligands specific for target cell attachment, retention, spreading and/or differentiation.

In certain embodiments, the activation of a surface of the non-biodegradable prosthesis is achieved by etching the surface and removing at least some of the ions attached thereto. This activation makes the surface of the prosthesis more, hydrophilic, thereby allowing improved interaction of the polar adhesion of a target cell ligand coating. Activation of the prosthesis surface may be achieved by any method known to persons skilled in the art, such as by treating the prosthesis surface with a strong acid or base, or by using plasma glow discharge. Preferably, plasma glow discharge is used to activate the prosthesis surface by placing the prosthesis within the vacuum chamber of a plasma glow discharge device such as an EMS-100 Glow Discharge Unit (Electron Microscopy Services, Inc., Ft. Washington, Pa.) for an exposure to at least about 10 to about 50 mAmps cathode positive charge direct current for at least about 1 to about 10 minutes. The prosthesis is placed within the vacuum chamber of the plasma glow discharge for an exposure to at least about 25 to about 35 mAmps cathode positive charge direct charge for at least about 3 to about 6 minutes. After activation, the prosthesis is ready to be coated with the adhesion target cell ligand coating.

In other embodiments, the target cell ligand is integrated into the material of the prosthetic device and presented on the blood contacting surface, eliminating the need for a ligand coating on the blood contacting surface. If, for example, the material of the prosthesis is made from a polymer containing a particular amino acid sequence, such as the RGD sequence, which represents a binding sequence within extracellular matrix proteins for cellular integrins, these particular sequences would not only be present in the bulk of the material but also at the prosthesis surface where they can interact with the surface receptors of the target cells.

In other embodiments, after the prosthesis is activated, a polymer layer is applied to the blood contacting surface of the prosthesis to be selected to be cellularized. A blood contacting surface coating may comprise a layer or plurality of layers of a polymer such as poly(2-hydroxyethylmethacrylate) and other polymers with functional groups. The polymer layer may be hydrophobic or hydrophilic depending on the molecules to be attached to the coating on the blood contacting surface of the prosthesis.

In certain embodiments, the surface coating is a polymer layer comprised of a hydrogel which absorbs water and provides a high number of available hydroxyl groups to facilitate the binding of a coating comprising a target cell ligand such as an adhesion peptide (EPC ligand) The polymer layer may comprise an acrylic resin, such as polymers and co-polymers of acrylic acid, methacrylic acid, esters of these acids, or acrylonitrile; methacrylates; polyvinyl alcohols; and glycophase. Exemplary polymer surface layers for activating the surface 6f a non-biodegradable prostheses are 2-hydroxyethlmethacrylate (HEMA) or poly(2-hydroxyethylmethacrylate) (polyHEMA).

The polymer layer may be formed on the blood contacting surface by applying any suitable polymer to the blood contacting surface by any method known to persons skilled in the part. In certain embodiments, the polymer layer is applied to the surface of the prosthesis in liquid form and allowed to dry leaving polymer layer attached to the scaffolding surface. For example, in one embodiment, 1% polyHEMA methanol solution is applied to the prosthesis surface. The prosthesis is allowed to dry, i.e., the methanol evaporates away from the prosthetic scaffolding, leaving behind a polymer layer consisting of polyHEMA.

Any method known to persons skilled in the art may be used to facilitate drying, e.g., allowing the prosthesis to dry naturally, i.e., air dry, at room temperature for a time sufficient for the polymer layer to dry. In certain embodiments, the polymer layer on the prosthesis is allowed to dry at 50° C. for 30 minutes. A plurality of polymer layers may be applied to the prosthesis following the same steps as described above. After the surface of the prosthesis is activated, molecules (ligands) which bind or interact with target cells may be attached to the activated blood contacting surface of the prosthesis.

In other embodiments, the initial polymer layer comprises the target cell ligands reducing the number of coatings applied to the blood contacting surface. The polymer layer may also comprise target cell modifying agents to induce cellular spreading and/or differentiation of target cells on the blood contacting surface. The polymer layer in certain embodiments comprises compounds which allow for the time release of modifying agents over a period of time. Exemplary time release coatings include calcium alginate, polylactic acid, coatings containing elements subject to hydrolytic cleavage such as chitosan, or protein coatings which are subject to enzymatic degradation such as collagen. The substance which is released from the coating over time may be embedded or encapsulated within the coating. In certain instances, the substance may be coupled to the ligand and released over time from its binding partner, e.g. VEGF or FGF from heparin, where the lifetime of the molecular bond will determine the release kinetics. The modifying agents may be released over a duration of about 1 day to about 90 days.

Biodegradable Prostheses/Scaffolding

The biodegradable prostheses or scaffolding in the case of forming a neo-endothelium of the invention are made from biodegradable materials which degrade at a rate that allows the prosthesis/scaffolding to accommodate the mechanical load of blood flow passing through the scaffolding until the vascular tissue around the exterior of the scaffolding has developed sufficiently to withstand the physiological pressures associated with blood flow. This time-frame of scaffolding degradation may range, for example, from approximately 1 week to approximately 12 months, typically from 2 weeks to 4 months and more typically from 1 month to three months, depending upon the size and nature of the blood vessel into which the scaffolding is implanted. In addition to appropriate degradation times, the biodegradable materials useful in the present invention provide elasticity to the scaffolding—either by choice of material and/or by appropriate processing (e.g., weaving/knitting structure). These biomaterials also allow for the stress-conditioning of the encapsulating tissue which helps to prevent aneurysms from forming in the neo-vascular structure after the scaffolding has degraded.

Suitable materials for a polymeric biodegradable scaffolding include, but are not limited to, polyglycolide (PGA), copolymers of glycolide, glycolide/L-lactide copolymers (PGA/PLLA), lactide/trimethylene carbonate copolymers (PLA/TMC), glycolide/trimethylene carbonate copolymers (PGA/TMC), polylactides (PLA), stereo-copolymers of PLA, poly-L-lactide (PLLA), poly-DL-lactide (PDLLA), L-lactide/DL-lactide copolymers, copolymers of PLA, lactide/tetramethylglycolide copolymers, lactide/α-valerolactone copolymers, lactide/α-caprolactone copolymers, hyaluronic acid and its derivatives, polydepsipeptides, PLA/polyethylene oxide copolymers, unsymmetrical 3,6-substituted poly-1,4-dioxane-2,5-diones, poly-β-hydroxybutyrate (PHBA), PHBA/β-hydroxyvalerate copolymers (PHBA/HVA), poly-p-dioxanone (PDS), poly-α-valerlactone, poly-β-caprolactone, methacrylate-N-vinyl-pyrrolidone copolymers, polyesteramides, polyesters of oxalic acid, polydihydropyranes, polyalkyl-2-cyanoacrylates, polyurethanes, polyvinylalcohol, polypeptides, poly-B-malic acid (PMLA), poly-B-alcanoic acids, polybutylene oxalate, polyethylene adipate, polyethylene carbonate, polybutylene carbonate, tyrosine based polycarbonates, chitin derivates such as chitosan and other polyesters containing silyl ethers, acetals, or ketals, alginates, and blends or other combinations of the aforementioned polymers. In addition to the aforementioned aliphatic link polymers, other aliphatic polyesters may also be appropriate for producing aromatic/aliphatic polyester copolymers. These include aliphatic polyesters selected from the group of oxalates, malonates, succinates, glutarates, adipates, pimelates, suberates, azelates, sebacates, nonanedioates, glycolates, and mixtures thereof. These materials are of particular interest as biodegradable support members in applications requiring temporary support during tissue or organ regeneration. The synthesis and formulation of biodegradable implant compositions for selected mechanical properties are well known to those skilled in the art, and the aforementioned materials may be utilized to prepare scaffolding compositions suitable for use with the invention.

More particularly, suitable biodegradable materials for a prosthesis or the support member of the scaffolding of the present invention include polylactic acid, polyglycolic acid (PGA), collagen or other connective proteins or natural materials, polycaprolactone, hylauric acid, adhesive proteins, co-polymers of these materials as well as composites and combinations thereof and combinations of other biodegradable polymers. Biodegradable glass or bioactive glass is also a suitable biodegradable material for use in the present invention.

Properties of a Biodegradable Prosthesis/Scaffolding

The polymeric material of the scaffolding is designed and configured to achieve a controlled permeability, either for control of materials within the cellularized (e.g. endothelialized) surface or for release of incorporated materials. For exemplary purposes, the following description of the properties of the biodegradable prosthesis is disclosed in terms of the target cells being endothelial progenitor cells. There are basically two functions of the polymeric material: the passage of only nutrients (small molecular weight compounds), and the passage of gases from the scaffolding surface exposed to circulating blood through the polymeric material to the vascular cells growing on a non-endothelialized (exterior) surface of the scaffolding. The permeability and molecular weight ranges of the aforementioned materials are known and can therefore be used to determine the desired porosity. For example, a macromolecule can be defined as having a molecular weight of greater than 1000 daltons; cells generally range from about 600-700 nm to 50 microns, with aggregates of from about 30 to 150 microns in size.

The permeability of the biodegradable scaffolding, and in particular the support member, is sufficient to allow gases and nutrients to flow between the vascular cells on a non-endothelialized surface of the scaffolding and the growing endothelium on the endothelialized surface of the scaffolding, allowing the formation of the vascular structure and the neoendothelium without sacrificing the integrity of the scaffolding in regards to withstanding the mechanical load associated with blood flow. In one embodiment of the present invention, the scaffolding is microporous and constructed of biodegradable polymeric material that is permeable to the bulk flow of macromolecules, but impermeable to cellular in growth and is of sufficient strength to serve mechanically as a blood vessel substitute during the formation of the neo-vessel.

The permeability characteristics of the scaffolding can be evaluated using markers of known sizes, such as dextrans and polystyrene microspheres. For example, dextran, labeled with fluorescein (Sigma Chemical Co., St. Louis, Mo.), with an average molecular weight of about 2,000,000 MW can be used to test the ability of the scaffolding to pass macromolecules. Cell impermeability of the scaffolding and in particular the support member of the scaffolding, can be tested using polystyrene microspheres (Polysciences, Inc., Warrington, Pa.), with a diameter of about 3 μm at a concentration of about 2.5% solids in suspension, for example.

An appropriate scaffolding material, in certain embodiments, will pass about 2,000,000 MW dextran at pressures at or below about 20.7 kPa so that the solution that filters through the sheath is visibly colored when viewed against a white background.

Additionally, the sheath will not allow more than about 5% of the 3 μm microspheres to pass at a pressure of about 20.7 kPa.

The biodegradable scaffolding of the present invention incorporates a variety of biodegradable materials within it or coated on the surface of a support member. For instance, in one embodiment, the main body or support member of the biodegradable scaffolding is made of either polylactic acid, polyglycolic acid (PGA), collagen or other connective proteins or natural materials, polycaprolactone, copolymers of these materials as well as composites thereof and combinations of other biodegradable polymers. The layer covering the exterior scaffolding surface may be made of either collagen, hylauric acid, adhesive proteins, copolymers of these materials as well as composites and combinations thereof which enhance the development of a exterior vascular structure and/or strengthen the scaffolding. Also, the present invention includes an embodiment where the film or layers (both outer and lumen layers) are made from a biodegradable material different from the support member.

Factors that effect the rate of degradation of the biopolymers are increased hydrophilic backbone, more hydrophilic end-groups, more reactive hydrolytic groups in the backbone, less crystallinity, more porosity, and the size of the device. For porous support members comprised of hydrophobic biodegradable polymer based materials, the present invention also permits ligands to be readily immobilized on the surfaces defining the porous regions of the support member without significantly reducing the porosity of the support member. The result is a biodegradable support member having surfaces rendered hydrophilic and wetable with high surface tension fluids throughout its bulk to which at least one type of ligand (i.e., EPC ligand) is immobilized.

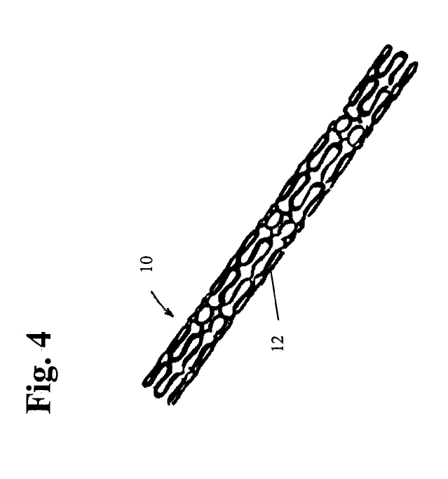

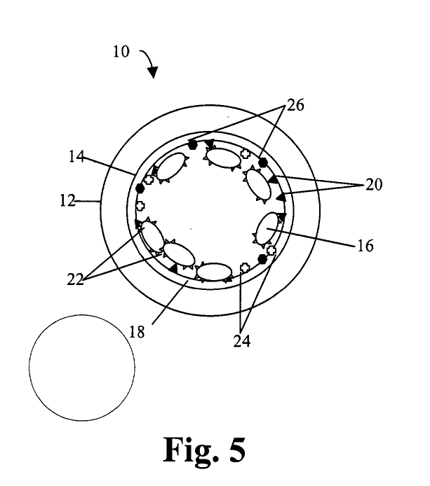

In certain embodiments, the prosthesis of the present invention is a stent comprising a blood contacting surface when implanted into a subject. FIG. 4 and FIG. 5 are a plan view and a cross-section view, respectively of a non-degradable stent of the present invention. The non-degradable stent 10, comprises an exterior surface 12, and a blood contacting (lumen surface) 14 which is exposed to the bloodstream of a subject when the stent 10 is positioned into the vascular circulatory system of a subject. The blood contacting surface 14 is configured to form a bond with target cells 16 circulating in the blood stream. Blood contacting surface 14 comprises a coating 18, wherein the coating comprises ligands 20 which attract and bind the circulating target cells 16 from the bloodstream of the subject. The ligand 20 on the blood contacting surface 14 is a ligand for the cell surface receptor 22 on the target cell 16. As shown in FIG. 5, the coating 18 in this embodiment of the device of the present invention, further comprises a target cell modifying agent 24 which induces the differentiation of target cells 16 over the blood contacting surface 14. The coating 18, further comprises at least one compound 26 for decreasing the adhesion of cells other than the target cell. The compound 26 for decreasing the adhesion of unwanted cell types, comprises compounds that are hydrophilic polymers such as polyethylene glycols, phosphoryl choline based polymers, phosphatidyl choline based polymers, alginic acid polymers and poly vinyl pyrrolidon. After the binding of the target cell 16 to the ligand 20 on the blood contacting surface 14, the target cells spread and differentiate to provide a cellularized surface over the blood contacting surface 14 of the stent 10.

### Kits

Also provided are kits for use in practicing the subject methods. The kits of the subject invention may comprise, for example, a coating comprising a ligand specific for a circulating target cell, the coating configured to be layered on a blood contacting surface in vivo or ex vivo. The kits may further comprise cultured target cells comprising a binding partner molecule on the cell surface to the ligand in the coating. The kits may further comprise printed instructions for application of a bodily fluid to the test strips, and use of the reader or meter for measuring values from the test strips.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Recruitment of Endothelial Progenitor Cells to a Blood Contacting Surface In Vivo by Ligand Interaction

As an exemplary method for the recruitment of circulating progenitor cells, e.g. endothelial progenitor cells, to a prosthesis surface via magnetic interaction, details of endothelial progenitor cell recruitment to a stent surface are provided. Antibodies to the CD34 receptor found on circulating progenitor cells, or alternatively to the CD133 receptor, are immobilized on the surface of a nitinol stent. When progenitor cells carrying the CD34 membrane receptor (or the CD133 receptor) come into contact with the stent surface, the antibody binds its respective receptor, thereby recruiting the cell to the stent.

In order to immobilize the antibodies to the stent surface, first, a carboxyl terminated polyethylene glycol spacer is conjugated to the nitinol by gamma irradiation. After cleaning the nitinol in a 1% sodium dodecylsulfate solution and drying the nitinol surface, it is immersed in a solution of 5% trichlorovinylsilane in chloroform at room temperature. After a three hour incubation they are rinsed in chloroform, ethanol and finally deionized water before being dried at 60C for five hours. The nitinol surface is then incubated in a 5 mg/ml solution of carboxyl terminated poly ethylene glycol polymers, and subsequently radiated by a dose of 1 Mrad. Excess unbound polymer is washed away by repeated rinses in phosphate buffered saline. In a final conjugation step, the CD34 (or CD133) antibody is conjugated to the carboxyl end of the polyethylene glycol by incubation in a 0.1 mg/ml antibody solution in phosphate buffered saline and 0.1mg/ml ethyl-dimethylaminopropyl carbodiimide (EDC). After this final conjugation step, the surface is rinsed and now presents antibodies to progenitor cell membrane receptors at its blood contacting surface.

### Example 2

### Recruitment of Endothelial Progenitor Cells to a Blood Contacting Surface of a Prosthesis via Magnetic Interaction In Vivo

As an exemplary method for the recruitment of circulating progenitor cells, e.g. endothelial progenitor cells, to a prosthesis surface via magnetic interaction, details of endothelial progenitor cell recruitment to the lumen surface of a synthetic vascular graft are provided. A vascular ePTFE graft is modified by replacing ring segments of the vascular graft by samarium cobalt rings, which are gold coated and magnetized to saturation in an annular fashion. Immediately prior to implantation of the graft prosthesis, the recipient is administered 200 nm ferrite particles which are encapsulated in a crosslinked gelatin matrix and present CD34 antibodies at their surface. CD34 antibodies are conjugated to the surface of these encapsulated particles through the crosslinking agent ethyl-dimethylaminopropyl carbodiimide (EDC). This conjugation takes place at 25C in amine-free buffer of pH 6 containing 1 mg/ml antibody and 1 mg/ml EDC. Antibodies are conjugated to the surface in random orientation, providing at least some binding sites for CD34 receptors at the particle surface. Approximately 10⁸ of these particles, dispersed into 10 ml saline, are intravenously injected into recipient. When these beads come into contact with CD34 positive progenitor cells, they will attach to the surface of these cells, and thus magnetically label them.

When the cells come into the proximity of the magnetic ring segments of the vascular graft, the cell-attached particles are attracted to these ring segments, thereby recruiting the cells to the lumen surface of the graft.

Alternatively, the pores of the ePTFE graft may be filled with fibrin glue containing 5 μM magnetic spheres, which are magnetized to saturation. This will result in focal magnetic attractors on the lumen surface of the vascular prosthesis, serving as points of recruitment for magnetically labeled circulating cells.

### Example 3

### Recruitment of Surface Modified Cells to a Blood Contacting Surface

As an exemplary method for the recruitment of surface modified cells to a cellularized heart valve prosthesis surface via receptor-ligand interaction, details of the recruitment of surface modified bone marrow cells to a prosthesis surface are provided.

Bone marrow cells are harvested from the bone marrow by punctation of the bone, or by aspirating bone marrow from a dissected bone with a syringe during surgery. Bone marrow cells are purified by density gradient centrifugation in Ficoll of density 1.077 at 400 g for 30 minutes. Bone marrow cells are modified at their surface through conjugation of hydroxysuccinimide-poly ethyleneglycol-biotin of molecular weight 3400 to amine groups of cell membrane proteins. Conjugation is carried out in a protein-free buffer of pH 8.5, i.e. a 150 mM carbonate-bicarbonate buffer, containing 2 mg/ml of the biotinylated polymer. Cells are incubated in this solution for 20 minutes at 37C. After the incubation, the solution is replaced with Medium 199 containing 10% autoiogous serum. After this labeling, the cells present biotin, which is known for its high affinity for avidin, at their surface. To recruit the biotin-labeled cells, avidin is immobilized at the prosthesis surface. This is achieved by conjugating hydroxysuccinimide-poly ethyleneglycol-biotin of molecular weight 3400 to the heart valve surface by incubation in a 150 mM carbonate-bicarbonate buffer of pH 8.5 containing 5 mg/ml of the biotinylated polymer for 30 minutes at 25C.

After washing the heart valve in physiological phosphate buffered saline, the valve prosthesis is incubated in phosphate buffered saline containing 100 μg/ml streptavidin. Streptavidin, an avidin isoform has four binding sites, two each on opposing sides. Thus, while being bound to surface-immobilized biotin on one side, avidin presents empty binding sites on the blood contacting side. When cells come into contact with the blood contacting surface after injection into the blood stream, the cell surface labeling biotin binds to the streptavidin on the prosthesis surface, thus retaining the cells on the surface.

### Example 4

### Exterior Vascular Tissue Formation

Animal experiments were conducted to study the generation of small diameter vascular conduits in a relatively short period of time that would support blood flow without leakage. The experiments were completed in high flow conditions for testing the hemostatic capability of the synthetic grafts. Synthetic mandrels (scaffolding), formed of silicone and utilizing the same external surface chemistry and morphology, were implanted in five swine weighing between 40 and 50 kg.

The scaffoldings were implanted between the aorta and right atrium. In each animal a scaffolding was inserted within the pericardial sack and ran from the animal's aorta to the right atrium. In one of the animals, a second scaffolding ran from the aorta to the right ventricle. Implants were done surgically on the beating heart through a thoracotomy incision.

Angiograms taken one hour before sacrifice demonstrated excellent patency, despite the fact that experimental animals had not received either heparin or antiplatelet therapy at any point in the study. The scaffoldings became encapsulated by vascular tissue within three weeks. The scaffoldings were removed from the newly formed vascular structure at approximately the third-week interval, allowing blood to flow freely through the new conduit.

Both angiographic and surgical findings confirmed that the engineered vascular structure was patent and hemostatic. Histological data on the newly-formed vascular structure demonstrated a highly vascular, fibrous capsule that appeared to be similar to a natural vessel.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

### Example 5

### Neo-Vessel Formation Around a Biodegradable Scaffolding

The present invention provides devices and methods for engineering a self-endothelializing graft in situ by recruitment of circulating endothelial progenitor cells (EPCs) from the blood stream to the internal lumen surface of a biodegradable scaffolding, the formation of a adventitial structure on the scaffold's exterior surface, and the biodegradation of the scaffolding to leave a complete, functional vascular structure capable of supporting arterial pressures.

In one embodiment, the biodegradable scaffolding comprises a support member that is substantially made from a poly(glycolic acid) mesh, where the lumen surface of the support member is coated with a first layer comprising reversibly cross-linked polylysine to allow the attachment of a second layer comprising EPC binding ligands such as CD34 antibodies or CD34 antibody binding fragments thereof. The exterior surface of the support member may be coated with an outer layer of biodegradable materials capable of withstanding the mechanical load generated from the blood flow and to help prevent any blood leakage out of the exterior surface of the support member during the time the neo-vessel is formed. The degradation of the biodegradable scaffolding occurs simultaneously with the formation of a neo-endothelium and the encapsulation of the exterior scaffolding by vascular cells.

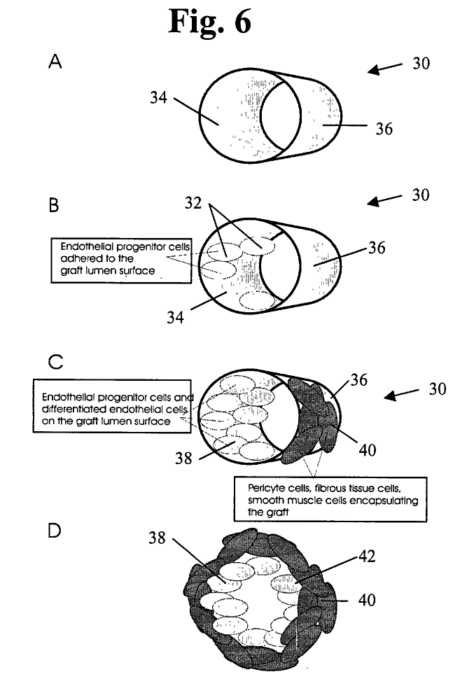

This invention pertains to devices and methods for developing endothelialized structures in situ that are suitable for use as artificial blood vessels. FIG. 6 shows a schematic drawing of one embodiment of the present invention as well as the recruitment, retention and spreading and the differentiation of EPCs into a functioning endothelium. The invention allows for the establishment of a permanent, bioactive, anti-thrombogenic graft 30 by the in vivo recruitment of endothelial progenitor cells (EPC) 32 in the blood of a graft recipient to the inner surface (i.e., the lumen surface) 34 of a biodegradable scaffolding 36 or support member, followed by the subsequent retention, spreading and differentiation of the adhered endothelial progenitor cells to allow the formation of a functioning endothelium, FIG. 6, section A, (naked graft short after implantation) and section B (endothelial progenitor cells are recruited from the blood stream). Simultaneously to the formation of a viable neo-endothelium 38, vascular tissue 40 forms around and encapsulates the exterior of the scaffolding 36, FIG. 6, section C. As such, the internal endothelium 38 and the external vascular tissue 40 form the respective inner and outer layers of a neo-vessel 42, FIG. 6, section D.

If biodegradable material is used as a scaffold, the engineered tissue will begin supporting the mechanical load as the synthetic material degrades. After degradation is completed, a non-synthetic neo-vessel will remain.

The biodegradable scaffolding is formulated to maintain sufficient structural integrity to perform the functions of a temporary vessel graft while the exterior tissue layer and the inner endothelial layer are being formed to create a neo-vessel. Substantial degradation/resorption of the biodegradable scaffolding occurs after sufficient encapsulation by pressure-resistant fibrous and smooth muscle tissue has occurred to allow the newly formed vascular tissue to carry the mechanical load associated with blood flow. Once the scaffolding has been biodegraded and resorbed, the resultant structure is a purely biological, stress-resistant vessel with a bioactive, anti-thrombogenic lumen surface.

The biodegradable scaffolding is formulated to maintain sufficient structural integrity to perform the functions of a temporary vessel graft while the exterior tissue layer and the inner endothelial layer are being formed to create a neo-vessel. Substantial degradation/resorption of the biodegradable scaffolding occurs after sufficient encapsulation by pressure-resistant fibrous and smooth muscle tissue has occurred to allow the newly formed vascular tissue to carry the mechanical load associated with blood flow. Once the scaffolding has been biodegraded and resorbed, the resultant structure is a purely biological, stress-resistant vessel with a bioactive, anti-thrombogenic lumen surface.

The biodegradable material for the scaffolding may comprise polylactide, polyglycolide, polylysine, or other biodegradable polymeric material, as well as copolymers and/or blends thereof. The scaffolding may be implanted at a selected location in association with a blood vessel via endoscopic surgical techniques. The biodegradable scaffolding is configured to operate as a temporary vascular graft capable of supporting arterial blood flow during the period in which vascular tissue forms on the exterior surface of the scaffolding. The biodegradable/resorbable material of the scaffolding is chosen to degrade under in vivo conditions within a selected time-frame that allows for the scaffolding to maintain adequate structural integrity while permitting encapsulation of the exterior of the scaffolding by pressure-resistant fibrous and smooth muscle tissue. Once the scaffolding has biodegraded and been resorbed within the body, the resulting vascular structure is a purely biological vessel having a structure capable of withstanding physiological pressures and having a bioactive, anti-thrombogenic lumen surface capable of functioning as a native vessel.

The biodegradable scaffolding in the present invention allows for the in vivo generation of a vascular tissue around the scaffolding and, due to the biodegradability, the vascular tissue will ultimately replace the biodegradable scaffolding. The newly formed vascular structure has the structural integrity to support the mechanical load associated with blood flow.

That which is claimed is:

1. A method for recruitment of cells to a blood contacting surface in vivo, comprising;
(a) providing a blood contacting surface positioned in the blood stream of a subject, said blood contacting surface configured to recruit target cells circulating in the blood stream of said subject to said blood contacting surface; and
(b) recruiting said target cells to said blood contacting surface.

2. The method of claim 1, wherein said blood contacting surface comprises a surface of a prosthesis implanted into said subject.

3. The method of claim 1, wherein said recruiting comprises magnetically attracting said target cells to said blood contacting surface.

4. The method of claim 1, further comprising introducing ligands onto said blood-contacting surface, said ligands having an affinity for said target cells.

5. The method of claim 4, wherein said recruiting comprises recruiting said target cells to said ligands.

6. The method of claim 2, further comprising introducing ligands onto said blood-contacting surface, said ligands having an affinity for said target cells.

7. The method of claim 6, wherein said recruiting comprises recruiting said target cells to said ligands.

8. The method of claim 1, wherein said target cells comprise progenitor cells.

9. The method of claim 1, wherein said target cells are selected from the group consisting of progenitor cells, red blood cells, mononuclear cells, macrophages, immune cells, and platelets.

10. The method of claim 2, wherein said target cells comprise progenitor cells.

11. The method of claim 2, wherein said target cells are selected from the group consisting of progenitor cells, red blood cells, mononuclear cells, macrophages, immune cells, and platelets.

12. The method of claim 1, wherein said progenitor cells comprise endothelial progenitor cells.

13. The method of claim 2, wherein said progenitor cells comprise endothelial progenitor cells.

14. The method of claim 1, further comprising introducing said target cells into said bloodstream of said subject.

15. The method of claim 2, further comprising introducing said target cells into said bloodstream of said subject.

16. The method of claim 15, wherein said target cells comprise autologous cells.

17. The method of claim 15, wherein said target cells comprise donor cells.

18. The method of claim 15, wherein said target cells comprise cells harvested from bone marrow or fat tissue.

19. The method of claim 15, wherein said introducing said target cells comprises injecting said target cells into said bloodstream of said subject.

20. The method of claim 4, further comprising modifying said target cells to increase affinity of said ligands for said target cells.

21. The method of claim 1, further comprising modifying said target cells to express a substance after said cell has adhered to said blood contacting surface.

22. The method of claim 20, wherein said modifying said target cells comprises changing a magnetic property of said targeted cells.

23. The method of claim 20, wherein said modifying said target cells comprises genetically manipulating said target cells.

24. The method of claim 20, wherein said modifying said target cells is carried out in vivo.

25. The method of claim 20, wherein said modifying said target cells is carried out in vitro.

26. The method of claim 24, wherein said modifying said target cells comprises injecting a modifying compound into said bloodstream of said subject.

27. The method of claim 1, further comprising increasing concentration of said target cells in said blood stream.

28. The method of claim 27, wherein said increasing concentration of said target cells comprises mobilizing said target cells from a tissue into said bloodstream, said tissue selected from the group consisting of bone marrow and fat tissue.

29. The method of claim 27, wherein said increasing concentration of said target cells comprises introducing said target cells into said bloodstream of said subject.

30. The method of claim 28, wherein said mobilizing of said target cells comprises subjecting said subject to at least one protein selected from the group consisting of FGF, VEGF, G-CSF, and GM-CSF.

31. The method of claim 14, wherein said introducing said target cells comprises using a catheter.

32. The method of claim 14, wherein said introducing said target cells is by an intravascular injection.

33. The method of claim 14, wherein said introducing said target cells is by diffusion of said targeted cells in to said bloodstream.

34. The method of claim 4, wherein said introducing said ligand comprises coating said ligand onto said blood contacting surface.

35. The method of 34, wherein said coating of said blood contacting surface is carried out in vivo.

36. The method of claim 2, wherein said coating further comprises introducing a layer of polymeric compound onto said blood contacting surface.

37. The method of claim 36, wherein said polymeric compound is cross-linked with a crosslinking agent that forms covalent bonds capable of enzymatic cleavage under in vivo conditions.

38. The method of claim 36, wherein said polymeric compound is cross-linked with a crosslinking agent that forms covalent bonds that are capable of non-enzymatic hydrolysis under in vivo conditions.

39. The method of claim 37, wherein said cross-linking agent comprises a compound having at least two reactive functional groups selected from the group consisting of aldehydes, epoxides, acyl halides, alkyl halides, isocyanates, amines, anhydrides, acids, alcohols, haloacetals, aryl carbonates, thiols, esters, imides, vinyls, azides, nitros, peroxides, sulfones, maleimides, poly(acrylic acid), vinyl sulfone, succinyl chloride, polyanhydrides, succinimidyl succinate-polyethylene glycol, and succinimidyl succinamide-polyethylene glycol, amine reactive esters.

40. The method of claim 34, wherein said ligand is capable of binding to a surface molecule of said target cells, said surface molecule selected from the group consisting of CD34, CD133, polysaccharides, KDR, P-selectin, glycophorin, CD4, integrins, lectins, and cadherins.

41. The method of claim 34, wherein said ligand comprises a compound selected from the group consisting of enzymes, organic catalysts, ribozymes, organometallics, proteins, glycoproteins, peptides, polyamino acids, antibodies, nucleic acids, steroidal molecules, antibiotics, antimycotics, cytokines, carbohydrates, oleophobics, lipids, viruses, and prions.

42. The method of claim 34, wherein said ligand is a compound selected from the group consisting of enzymes, organic catalysts, ribozymes, organometallics, proteins, glycoproteins, peptides, polyamino acids, antibodies, nucleic acids, steroidal molecules, antibiotics, antimycotics, cytokines, carbohydrates, oleophobics, lipids, viruses, and prions.

43. The method of claim 34, wherein said coating of said blood contacting surface further comprises introducing onto said blood contacting surface at least one compound which promotes differentiation of said targeted cells on said blood contacting surface.

44. The method of claim 43, wherein said blood contacting surface comprises at least one compound capable of promoting EPC differentiation.

45. The method of claim 44, wherein said compound capable of promoting said EPC differentiation is released from said blood contacting surface over a period of time, said period of time ranging from about 1 day to about 90 days.

46. The method of claim 34, wherein said blood contacting surface further comprises at least one compound which promotes cell spreading or retention of said targeted cells.

47. The method of claim 34, wherein said coating said ligand comprises adsorbing said ligand onto said blood contacting surface.

48. The method of claim 34, wherein said coating further comprises introducing a protein onto said blood contacting surface, said protein capable of mobilizing said targeted cells, said protein releasable over a period of time.

49. The method of claim 34, wherein said coating comprises introducing a polymeric material onto said blood contacting surface, said polymeric material capable of providing controlled release over time of a protein capable of mobilizing said targeted cells.

50. The method of claim 49, wherein the period of time for releasing said protein from said coating on said blood contacting surface ranges from about 1 day to about 90 days after introducing said blood contacting surface into said blood stream of said subject.

51. The method of claim 49, wherein said compound released from said coating is selected from the group consisting of cytokines, growth factors, cytokine mimics and growth factor mimics.

52. The method of claim 4, wherein said ligands is magnetically charged.

53. The method of claim 52, further comprising modifying said target cells by introducing a magnetic particle to said target cells.

54. The method of claim 20, wherein said modifying said target cells comprises changing an electrostatic property of said cell.

55. The method of claim 1, further comprising altering a surface characteristic of said blood contacting surface by said target cells.

56. The method of claim 55, wherein altering of said blood contacting surface by said target cells facilitates the in vivo formation of a cellular tissue on said blood contacting surface.

57. The method of claim 56, wherein said cellular tissue is a tissue selected from the group consisting of endothelial, fibrous, epithelial, and bone tissue.

58. The method of claim 2, wherein said prosthesis is selected from the group consisting of a stent, an anastomotic device, a diagnostic device, a pacemaker, a heart valve, a vascular graft, a synthetic organ, an artificial heart, a prosthesis, a drug delivering pump, a graft, an autologous graft, a homograft, a xenograft, and a tissue engineered graft.

59. The method of claim 2, wherein said prosthesis comprises a graft.

60. The method of 58, wherein said graft is selected from the group of consisting of a blood vessel graft, an organ graft, a heart graft, a lung graft, and a kidney graft.

61. A prosthesis, comprising:
(a) a support member having an exterior surface and a blood contacting surface;
(b) a first layer of a cross-linked polymeric compound coated onto said blood contacting surface of said support member; and,
(c) a second layer coated on said first layer, said second layer comprising at least one ligand having an affinity for a targeted cell in vivo.

62. The prosthesis of claim 61, wherein said support member comprises a material selected from the group consisting of polyglycolide (PGA), copolymers of glycolide, glycolide/L-lactide copolymers (PGA/PLLA), lactide/trimethylene carbonate copolymers (PLA/TMC), glycolide/trimethylene carbonate copolymers (PGA/TMC), polylactides (PLA), stereo-copolymers of PLA, poly-L-lactide (PLLA), poly-DL-lactide (PDLLA), L-lactide/DL-lactide copolymers, copolymers of PLA, lactide/tetra-methylglycolide copolymers, lactide/α-valerolactone copolymers, lactide/ε-caprolactone copolymers, PLA/polyethylene oxide copolymers, poly-β-hydroxybutyrate (PHBA), PHBA/β-hydroxyvalerate copolymers (PHBA/HVA), poly-p-dioxanone (PDS), poly-α-valerolactone, poly-β-caprolactone, methylmethacrylate-N-vinyl-pyrrolidone copolymers, polyesters of oxalic acid, polyalkyl-2-cyanoacrylates, polyurethanes, polybutylene oxalate, polyethylene adipate, polyethylene carbonate, polybutylene carbonate, tyrosine based polycarbonates, polyesters containing silyl ethers, chitin derived polymers and blends of the aforementioned polymers.

63. The prosthesis of claim 61, wherein said cross-linked polymeric compound is crosslinked with a cross-linking agent having at least two functional groups selected from the group consisting of aldehydes, epoxides, acyl halides, alkyl halides, isocyanates, amines, anhydrides, acids, alcohols, haloacetals, aryl carbonates, thiols, esters, imides, vinyls, azides, nitros, peroxides, sulfones, maleimides, poly(acrylic acid), vinyl sulfone, succinyl chloride, polyanhydrides, succinimidyl succinatepolyethylene glycol, and succinimidyl succinamide-polyethylene glycol.

64. The prosthesis of claim 63, wherein said cross-linking agent is capable of forming covalent bonds that are subjected to enzymatic cleavage under in vivo conditions.

65. The prosthesis of claim 63, wherein said cross-linking agent is capable of forming covalent bonds that are subjected to non-enzymatic hydrolysis under in vivo conditions.

66. The prosthesis of claim 61, further comprising a spacer compound interposed between said first layer and said second layer of the surface of said support member.

67. The prosthesis of claim 66, wherein said spacer compound comprises a hydrophilic polymer.

68. The prosthesis of claim 66, wherein said spacer compound is selected from the group consisting of succinic acid, diaminohexane, glyoxylic acid, short chain polyethylene glycol, and glycine.

69. The prosthesis of claim 62, wherein the prosthesis is biodegradable.

70. The prosthesis of claim 61, further comprising a first layer coated onto said exterior surface of said support member, said first layer on said exterior surface comprising at least one polymeric compound configured to allow said prosthesis to withstand a mechanical load associated with in vivo blood flow.

71. The prosthesis of claim 61, wherein said ligand is capable of binding to endothelial progenitor cells.

72. The prosthesis of ciaim 71, wherein said ligand is capable of binding to an endothelial progenitor cell surface molecule selected from the group of CD34, CD133, KDR (VEGFR-2), VE-Cadherin, E-selectin, αᵥβ₃ and lectins.

73. The prosthesis of claim 72, wherein said ligand comprises a compound capable of binding to CD34 receptors on endothelial progenitor cells.

74. The prosthesis of claim 72, wherein said ligand is a compound capable of binding to CD133 receptors.

75. A method for generating a self-endothelializing graft in vivo, the method comprising:
(a) providing a scaffolding configured to function as a vascular graft, said scaffolding having a lumen surface and exterior surface, said lumen surface comprising ligands specific for binding to endothelial progenitor cells;
(b) implanting said scaffolding into a blood vessel of a subject; and
(c) recruiting circulating endothelial progenitor cells to said lumen surface of said scaffolding to form a neo-endothelium.

76. The method of claim 75, wherein said scaffolding is biodegradable.

77. The method of claim 75, further comprising encapsulating said exterior surface of said scaffolding by vascular tissue to form an exterior hemostatic vascular structure.

78. The method of claim 75, wherein said ligands bind to endothelial progenitor cell surface molecules selected from the group of CD34, CD133, KDR (VEGFR-2), VE-Cadherin, E-selectin, αᵥβ₃ or lectins.

79. The method of claim 75, wherein said ligands comprise CD34 antibodies.

80. The method of claim 75, wherein said ligands comprise CD133 antibodies.

81. The method of claim 75, wherein said lumen surface further comprises at least one compound which promotes EPC differentiation into endothelial cells, said compound being selected from one or more of the group consisting of vascular endothelial growth factor, fibroblast growth factor and stem cell factor.

82. The method of claim 75, wherein said lumen surface further comprises at least one compound which promotes endothelial cell spreading or retention, said compound selected from the group consisting of Arg-Gly-D, Arg-Glu-D-Val, fibrin, fibronectin, laminin, gelatin, collagen, basement membrane proteins, and partial sequences of fibrin, fibronectin, laminin, gelatin, collagen, and basement membrane proteins.

83. The method of claim 76, wherein said degrading of said biodegradable scaffolding is controlled by making said scaffolding from a biodegradable material having a selected degradation rate under in vivo conditions.

84. The method of claim 76, wherein said biodegradable material is selected from the group consisting of polyglycolide, polylactide, polycaprolactone, p-dioxanone, polyanhydrides, polyothroesters, polylysine, tyrosine based polycarbonates, trimethylene carbonate, and copolymers and blends thereof.

85. The method of claim 75, wherein said recruiting of said circulating endothelial progenitor cells further comprises administering a compound to said subject in an effective amount that increases concentration of said endothelial progenitor cells in said bloodstream of said subject.

86. The method of claim 75, wherein said recruiting of said circulating endothelial progenitor cells further comprises mobilizing said progenitor cells by increasing blood serum level of a substance selected from the group consisting of VEGF, GM-CSF, and G-CSF.

87. A method for generating a self-endothelializing graft in situ, the method comprising:
(a) providing a prosthetic structure having a surface exposed to circulating blood;
(b) implanting the prosthetic structure into a subject; and
(c) recruiting circulating endothelial progenitor cells (EPCs) from the blood to the surface of the prosthetic structure to form a neo-endothelium thereon.

88. The method of 87, further comprising encapsulating an exterior surface of the scaffolding by vascular tissue to form an exterior hemostatic vascular structure.

89. The method of claim 87, wherein the lumen surface is modified to comprise a ligand specific for binding the endothelial progenitor cells to the lumen surface of the scaffolding.

90. The method of claim 21, wherein said modifying said target cells comprises genetically manipulating said target cells.

91. The method of claim 21 ,wherein said modifying said target cells is carried out in vivo.

92. The method of claim 21, wherein said modifying said target cells is carried out in vitro.

93. A method for generating a self-endothelializing graft in situ, the method comprising:
(a) providing a biodegradable scaffolding configured to function as a temporary vascular graft, the scaffolding having a lumen surface and exterior surface;
(b) implanting the biodegradable scaffolding into a blood vessel;
(c) recruiting circulating endothelial progenitor cells (EPCs) to the lumen surface of the biodegradable scaffolding to form a neo-endothelium;
(d) encapsulating the exterior surface of the scaffolding by vascular tissue to form an exterior hemostatic vascular structure; and
(e) degrading the biodegradable scaffolding under in vivo conditions within a time frame which allows the neo-endothelium and the exterior vascular structure to form a functional neo-vessel.

94. The method of claim 93, wherein the lumen surface is modified to comprise a ligand specific for binding the endothelial progenitor cells to the lumen surface of the biodegradable scaffolding.

95. The method of claim 94, wherein the ligand binds to an endothelial progenitor cell surface molecule selected from the group of CD34, CD133, KDR (VEGFR-2), VE-Cadherin, E-selectin, αᵥβ₃, or lectins.

96. The method of claim 94, wherein the ligand is a CD34 antibody.

97. The method of claim 94, wherein the lumen surface further comprises at least one compound which promotes EPC differentiation into endothelial cells, the compound being selected from one or more of the group comprising VEGF (vascular endothelial growth factor), FGF (fibroblast growth factor) and SCF (stem cell factor).

98. The method of claim 94, wherein the lumen surface further comprises at least one compound which promotes endothelial cell spreading or retention, the compound selected from the group of Arg-Gly-D, Arg-Glu-D-Val, fibrin, fibronectin, laminin, gelatin, collagen or basement membrane proteins.

99. The method of claim 93, wherein the degrading of the biodegradable scaffolding is controlled by the making of the scaffolding from a biodegradable material having a selected degradation under in vivo conditions.

100. The method of claim 93, wherein the degrading of the biodegradable scaffolding is controlled by the making of the scaffolding comprised of biodegradable material having a selected degradation under in vivo conditions, the biodegradable material being selected from the polymer group of polyglycolide, polylactide, polycaprolactone, p-dioxanone, polyanhydrides, polyothroesters, polylysine, tyrosine based polycarbonates, trimethylene carbonate, and copolymers and blends thereof.

101. The method of claim 93, wherein the recruiting of circulating endothelial progenitor cells comprises administering a compound to the graft recipient in an effective amount that increases the concentration of endothelial progenitor cells in the blood.

102. A biodegradable scaffolding for forming an endothelialized vascular graft in situ, the scaffolding comprising:
(a) a porous biodegradable support member having a lumen and an exterior surface; and
(b) the lumen surface comprising a first layer of at least one species of a polymeric compound coated to the support member, and wherein the compound is cross-linked to itself with a cross-linking agent that forms covalent bonds that are subject to enzymatic cleavage or non-enzymatic hydrolysis under in vivo conditions.

103. The biodegradable scaffolding of claim 102, wherein the exterior surface comprises a first layer coated to the support member, the first layer of the exterior surface comprising at least one polymeric compound which allows the biodegradable scaffolding to withstand the mechanical load associated with in situ blood flow.

104. The biodegradable scaffolding of claim 102, wherein the support member is selected from a member of the groups consisting of polyglycolide (PGA), copolymers of glycolide, glycolide/L-lactide copolymers (PGA/PLLA), lactide/trimethylene carbonate copolymers (PLA/TMC), glycolide/trimethylene carbonate copolymers (PGA/TMC), polylactides (PLA), stereo-copolymers of PLA, poly-L-lactide (PLLA), poly-DL-lactide (PDLLA), L-lactide/DL-lactide copolymers, copolymers of PLA, lactide/tetra-methylglycolide copolymers, lactide/α-valerolactone copolymers, lactide/ε-caprolactone copolymers, PLA/polyethylene oxide copolymers, poly-β-hydroxybutyrate (PHBA), PHBA/β-hydroxyvalerate copolymers (PHBA/HVA), poly-p-dioxanone (PDS), poly-α-valerolactone, poly-ε-caprolactone, methylmethacrylate-N-vinyl-pyrrolidone copolymers, polyesters of oxalic acid, polyalkyl-2-cyanoacrylates, polyurethanes, polybutylene oxalate, polyethylene adipate, polyethylene carbonate, polybutylene carbonate, tyrosine based polycarbonates, polyesters containing silyl ethers and blends of the aforementioned polymers.

105. The biodegradable scaffolding of claim 102, wherein the cross-linking agent comprises a compound having at least two chemically functional groups selected from the group consisting of aldehydes, epoxides, acyl halides, alkyl halides, isocyanates, amines, anhydrides, acids, alcohols, haloacetals, aryl carbonates, thiols, esters, imides, vinyls, azides, nitros, peroxides, sulfones, and maleimides.

106. The biodegradable scaffolding of claim 102, wherein the cross-linking agent is selected from the group consisting of poly(acrylic acid), vinyl sulfone, succinyl chloride, polyanhydrides, succinimidyl succinate-polyethylene glycol, and succinimidyl succinamide-polyethylene glycol.

107. The biodegradable scaffolding of claim 102, further comprising a second layer of the lumen surface comprised of at least one ligand which binds to endothelial progenitor cells.

108. The biodegradable scaffolding of claim 107, wherein the ligand binds to an endothelial progenitor cell surface molecule selected from the group of CD34, CD133, KDR (VEGFR-2), VE-Cadherin, E-selectin, αᵥβ₃ or lectins.

109. The biodegradable scaffolding of claim 107, wherein the ligand is a compound which binds to CD34 receptors on endothelial progenitor cells.

110. The biodegradable scaffolding of claim 108, wherein a spacer compound is interposed between the first layer and the second layer of the lumen surface.

111. The biodegradable scaffolding of claim 110, wherein the spacer compound is selected from the group of succinic acid, diaminohexane, glyoxylic acid, short chain polyethylene glycol, and glycine.

112. A method for generating a self-endothelializing graft in situ, the method comprising:
(a) providing a scaffolding having a lumen surface and exterior surface;
(b) positioning the scaffolding in association with a blood vessel;
(c) recruiting circulating endothelial progenitor cells (EPCs) to the lumen surface of the scaffolding to form a neo-endothelium; and
(d) encapsulating the exterior surface of the scaffolding by vascular tissue to form an exterior hemostatic vascular structure.

113. A method for generating a self-endothelializing graft in situ, the method comprising:
(a) providing a prosthetic structure, having a surface exposed to circulating blood;
(b) implanting the prosthetic structure into a subject; and
(c) recruiting circulating endothelial progenitor cells (EPCs) from the blood to the surface of the prosthetic structure to form a neo-endothelium.

114. The method of claim 113, wherein the lumen surface is modified to comprise a ligand specific for binding the endothelial progenitor cells to the lumen surface of the biodegradable scaffolding.

115. A prosthesis for forming an endothelialized vascular graft in situ, the prosthesis comprising:
(a) a support member having a surface; and
(b) the surface comprising a first layer of at least one species of a polymeric compound coated to the support member, and wherein the compound is cross-linked to itself with a cross-linking agent that forms covalent bonds that are subject to enzymatic cleavage or non-enzymatic hydrolysis under in vivo conditions.

116. The prosthesis of claim 61, wherein said support member comprises a material selected from the group consisting of stainless steel, nitinol, titanium, gold, silicone, superelastic alloys, polytetrafluoroethylene, polyethylene terephthalate, polyesters, and polyethylenes.

117. The method of claim 14, wherein said target cells comprise autologous cells.

118. The method of claim 14, wherein said target cells comprise donor cells.

119. The method of claim 14, wherein said target cells comprise cells harvested from bone marrow or fat tissue.

120. The method of claim 14, wherein said introducing said target cells comprises injecting said target cells into said bloodstream of said subject.

121. The method of claim 1, wherein said coating further comprises introducing a layer of polymeric compound onto said blood contacting surface.

122. The method of claim 121, wherein said polymeric compound is cross-linked with a cross-linking agent that forms covalent bonds capable of enzymatic cleavage under in vivo conditions.

123. The method of claim 121, wherein said polymeric compound is cross-linked with a cross-linking agent that forms covalent bonds that are capable of non-enzymatic hydrolysis under in vivo conditions.

124. The method of claim 122, wherein said cross-linking agent comprises a compound having at least two reactive functional groups selected from the group consisting of aldehydes, epoxides, acyl halides, alkyl halides, isocyanates, amines, anhydrides, acids, alcohols, haloacetals, aryl carbonates, thiols, esters, imides, vinyls, azides, nitros, peroxides, sulfones, maleimides, poly(acrylic acid), vinyl sulfone, succinyl chloride, polyanhydrides, succinimidyl succinate-polyethylene glycol, and succinimidyl succinamide-polyethylene glycol, amine reactive esters.

125. A kit for recruiting target cells to a blood contacting surface comprising:
a coating comprising a ligand specific for a circulating target cell, and said coating configured to form a layer on a blood contacting surface in vivo.

126. The kit of claim 125, further comprising cultured target cells, said target cells comprising a binding partner molecule for said ligand.

127. The kit of claim 125, and instructions for using said kit.

128. A method for recruitment of cells to a blood contacting surface ex vivo, comprising;
(a) providing a blood contacting surface positioned in the blood stream of a subject, said blood contacting surface configured to recruit target cells circulating in the blood stream of said subject to said blood contacting surface; and
(b) recruiting target cells to said blood contacting surface.

129. The method of claim 2, further comprising altering a surface characteristic of said blood contacting surface by said target cells.

130. The method of claim 129, wherein altering of said blood contacting surface by said target cells facilitates the in vivo formation of a cellular tissue on said blood contacting surface.

131. The method of claim 130, wherein said cellular tissue is a tissue selected from the group consisting of endothelial, fibrous, epithelial, and bone tissue.
